(19) United States
(12) Patent Application Publication
(10) Pub. No.: US 2023/0144283 A1
(43) Pub. Date: May 11, 2023

(54) 8-SUBSTITUTED DIARYL XANTHINES AS DUAL A2A-A2B ANTAGONISTS

(71) Applicant: Inspyr Therapeutics,Inc., Westlake Village, CA (US)

(72) Inventor: Robert Thompson, Charlottesville, VA (US)

(21) Appl. No.: 17/907,096

(22) PCT Filed: Jan. 26, 2021
(86) PCT No.: PCT/US21/15087

§ 371 Date: Sep. 22, 2022

Related U.S. Application Data
(60) Provisional application No. 63/000,286, filed on Mar. 26, 2020.

(51) Int. Cl. C07D 473/06

(52) U.S. Cl. CPC C07D 473/06

(57) ABSTRACT

The present invention relates to 8-substituted diaryl xanthines and pharmaceutical compositions thereof that are antagonists of A_{2A} and/or A_{2B} adenosine receptors (ARs).

### FIELD OF THE INVENTION

The present invention relates to 8-substituted diaryl xanthines and pharmaceutical compositions thereof that are antagonists of A_{2A} and/or A_{2B} adenosine receptors (ARs). These compounds and compositions are useful as pharmaceutical agents.

### BACKGROUND OF THE INVENTION

Adenosine, a natural chemical in the body exhibits physiology effects in the human body through four different adenosine receptors, which are A₁, A_{2A}, A_{2B} and A₃. These receptors are subtypes of the G protein-coupled receptor. Extracellular adenosine accumulation, which can occur during acute injury, has been shown to have protective effects, which shield cells and tissues from an excessive inflammatory response and immune-mediated damage, and support a self-limiting immune response that aims to promote healing processes. Adenosine is involved in the regulation of proliferation, differentiation, and apoptosis of parenchymal cells. This is also true for cancer cells, as they are targets of the regulatory actions of adenosine. Thus, in addition to the effects of adenosine on the cancer stroma, which indirectly affect the course of cancer development, progression and metastasis are also determined by the direct effects of adenosine on cancer cells. Cancer cells utilize the ectonucleotidases CD39 and CD73 to metabolize ATP and ADP to AMP, and AMP to adenosine to generate the effects of suppressing the immune system through both adenosine A_{2A} and A_{2B} receptors, while activation of the A_{2B} receptor promotes angiogenic actions and tumor cell migration. Hence, developing an adenosine compound with the ability to antagonize both the A_{2A} and A_{2B} receptors, while keeping selectivity over the A₁ and A₃ receptors, would provide a useful approach to treating many different types of cancer.

Therefore, it would be beneficial to develop compounds that are antagonists of both A_{2A} and A_{2B} receptors.

### SUMMARY OF THE INVENTION

Accordingly, in an aspect, the present invention provides novel 8-substituted diaryl xanthines that are A_{2A}-A_{2B} antagonists.

In another aspect, the present invention provides novel pharmaceutical compositions, comprising: a pharmaceutically acceptable carrier and a therapeutically effective amount of at least one of the compounds of the present invention.

In another aspect, the present invention provides methods of treating a pathological condition or symptom in a mammal for which the A_{2A}-A_{2B} receptors are implicated and antagonism of the receptors provides therapeutic benefit by administering to a subject an effective amount of a compound of the present invention.

In another aspect, the present invention provides methods of treating an adenosine A_{2A}-A_{2B} receptor-associated state in a subject by administering to the subject an effective amount of a compound of the present invention.

In another aspect, the present invention provides compounds for use in medical therapy.

In another aspect, the present invention provides the use of compounds of the present invention for the manufacture of a medicament for the treatment of a pathological condition or symptom in a mammal for which the A_{2A}-A_{2B} receptors are implicated and antagonism of the receptor provides therapeutic benefit.

These and other objects, which will become apparent during the following detailed description, have been achieved by the inventors' discovery that the presently claimed compounds are expected to be effective A_{2A}-A_{2B} antagonists.

### DETAILED DESCRIPTION OF THE INVENTION

All references cited herein are hereby incorporated in their entirety herein by reference.

Wherever appropriate, when a compound(s) of the present invention is described or referenced, stereoisomers and/or pharmaceutically acceptable salt forms are also included.

In an aspect, the present invention provides novel compounds of Formula I or a stereoisomer or pharmaceutically acceptable salt thereof:

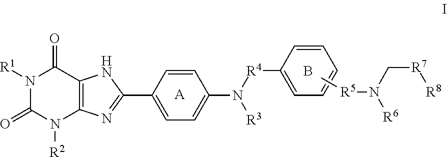

wherein:
Ring A is phenyl or a 5-6 membered heteroaryl having 1-3 heteroatoms selected from O, S, or N;
Ring B is phenyl or a 5-6 membered heteroaryl having 1-2 heteroatoms selected from O, S, or N;
R¹ is selected from: C₁₋₅ alkyl, —CH₂—C₂₋₄ alkenyl, —CH₂—C₂₋₄ alkynyl, C₃₋₆ cycloalkyl, and —C₂₋₅ alkylene-O—C₁₋₅ alkyl;
R² is selected from: C₁₋₅ alkyl, —CH₂—C₂₋₄ alkenyl, —CH₂—C₂₋₄ alkynyl, C₃₋₆ cycloalkyl, and —C₂₋₅ alkylene-O—C₁₋₅ alkyl;
R³ is selected from: H, C₁₋₅ alkyl, C₃₋₆ cycloalkyl, and —C₂₋₅ alkylene-O—C₁₋₅ alkyl;
R⁴ is —(CH₂)₁₋₆—;
R⁵ is —(CH₂)₁₋₆—;
R⁶ is selected from: H, C₁₋₅ alkyl, C₃₋₆ cycloalkyl, and —C₂₋₅ alkylene-O—C₁₋₅ alkyl;
R⁷ is absent or is selected from: C₁₋₅ alkyl, O, S, N—C₁₋₅ alkyl, and NH;
R⁸ is selected from: C₁₋₅ alkyl, —CH₂—C₂₋₄ alkenyl, —CH₂—C₂₋₄ alkynyl, C₃₋₆ cycloalkyl, —C₁₋₅ alkylene-O—C₁₋₅ alkyl, phenyl, 5-6 membered heterocycle, and 5-6 membered heteroaryl;
the phenyl and heteroaryl groups of R⁸ are optionally substituted with 1-3 groups independently selected from: C₁₋₄ alkyl, C₃₋₆ cycloalkyl, —C₁₋₃ alkylene-C₃₋₆ cycloalkyl, F, Cl, Br, I, —CN, OR^{a}, SR^{a}, NR^{a}R^{b}, CF₃, OCF₃, COR^{a}, CO₂R^{a}, C(O)NR^{a}R^{b}, OC(O)R^{a}, OCO₂R^{a}, OC(O)NR^{a}R^{b}, NR^{b}COR^{a}, NR^{b}CO₂R^{a}, NR^{b}C(O)NR^{a}R^{b}, and S(O)ₚNR^{a}R^{b};
each R^{a} is independently selected from: H, C₁₋₈ alkyl, C₃₋₆ cycloalkyl, and —C₁₋₃ alkylene-C₃₋₆ cycloalkyl;
each R^{b} is independently selected from: H, C₁₋₈ alkyl, C₃₋₆ cycloalkyl, and —C₁₋₃ alkylene-C₃₋₆ cycloalkyl;
alternatively, each NR^{a}R^{b} group is optionally selected from a 3-6 membered cyclic amine; and,
p is independently selected from: 0, 1, and 2.

In another aspect, the compound is of Formula IA:

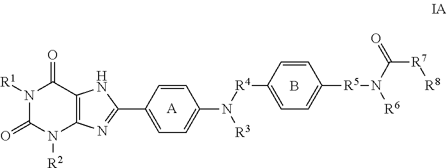

or a stereoisomer or pharmaceutically acceptable salt thereof.

In another aspect, the compound is of Formula IB:

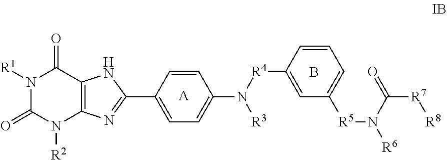

or a stereoisomer or pharmaceutically acceptable salt thereof.

In another aspect, the compound is of Formula IC:

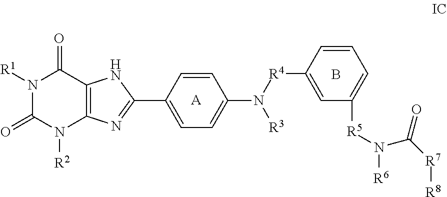

or a stereoisomer or pharmaceutically acceptable salt thereof.

In an aspect, the present invention provides novel compounds of Formula II or a stereoisomer or pharmaceutically acceptable salt thereof:

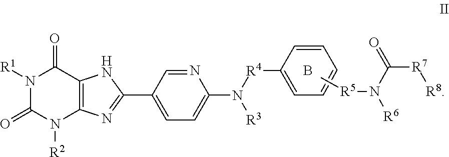

In another aspect, the compound is of Formula IIA:

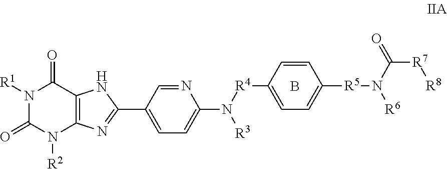

or a stereoisomer or pharmaceutically acceptable salt thereof.

In another aspect, the compound is of Formula IIB:

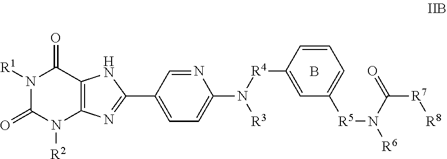

or a stereoisomer or pharmaceutically acceptable salt thereof.

In another aspect, the compound is of Formula IIC:

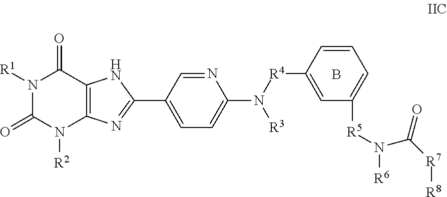

or a stereoisomer or pharmaceutically acceptable salt thereof.

In an aspect, the present invention provides novel compounds of Formula III or a stereoisomer or pharmaceutically acceptable salt thereof:

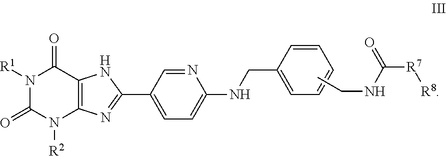

In another aspect, the compound is of Formula IIIA:

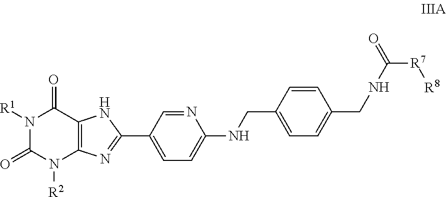

or a stereoisomer or pharmaceutically acceptable salt thereof.

In another aspect, the compound is of Formula IIIB:

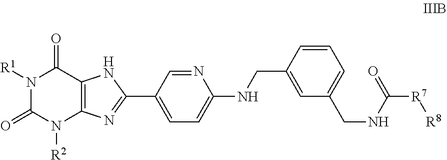

or a stereoisomer or pharmaceutically acceptable salt thereof.

In another aspect, the compound is of Formula IIIC:

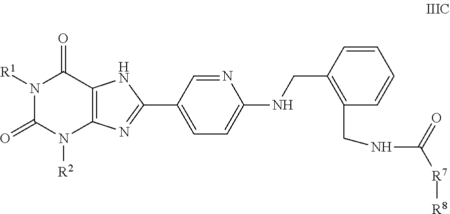

or a stereoisomer or pharmaceutically acceptable salt thereof.

In another aspect, the compound is of Formula I or a stereoisomer or pharmaceutically acceptable salt thereof, wherein:

Ring A is phenyl or a 6 membered heteroaryl having 1-2 heteroatoms selected from O, S, or N;
Ring B is phenyl or a 6 membered heteroaryl having 1 heteroatoms selected from N;
R¹ is selected from: C₁₋₃ alkyl, C₃₋₆ cycloalkyl, and —C₂₋₃ alkylene-O—C₁₋₂ alkyl;
R² is selected from: C₁₋₃ alkyl, C₃₋₆ cycloalkyl, and —C₂₋₃ alkylene-O—C₁₋₂ alkyl;
R³ is selected from: H and C₁₋₃ alkyl;
R⁴ is —(CH₂)₁₋₂—;
R⁵ is —(CH₂)₁₋₂—; and,
R⁶ is selected from: H and C₁₋₃ alkyl.

In another aspect, the compound is of Formula I or a stereoisomer or pharmaceutically acceptable salt thereof, wherein:

Ring A is phenyl or pyridyl;
Ring B is phenyl or pyridyl;
R¹ is selected from: ethyl, n-propyl, methoxyethylene, and cyclopropyl;
R² is selected from: ethyl, n-propyl, methoxyethylene, and cyclopropyl;
R³ is selected from: H and CH₃;
R⁴ is —(CH₂)₁₋₂—;
R⁵ is —(CH₂)₁₋₂—; and,
R⁶ is selected from: H and CH₃.

In another aspect, the compound is of Formula I or a stereoisomer or pharmaceutically acceptable salt thereof, wherein:

R⁷ is absent;
R⁸ is selected from: C₁₋₃ alkyl, C₃₋₆ cycloalkyl, —C₁₋₂ alkylene-O—C₁₋₂ alkyl, phenyl, and 5-6 membered heteroaryl;
the phenyl and heteroaryl groups of R⁸ are optionally substituted with 1-2 groups independently selected from: C₁₋₄ alkyl, —C₁₋₃ alkylene-C₃₋₆ cycloalkyl, F, Cl, OR^{a}, COR^{a}, and CO₂R^{a}; and,
each R^{a} is independently selected from: H and C₁₋₄ alkyl.

In another aspect, the compound is of Formula I or a stereoisomer or pharmaceutically acceptable salt thereof, wherein:

R⁷ is absent;
R⁸ is selected from: C₁₋₃ alkyl, C₃₋₄ cycloalkyl, —C₁ alkylene-O—C₁₋₂ alkyl, phenyl, thienyl, and pyridyl;
the phenyl and heteroaryl groups of R⁸ are optionally substituted with 1-2 groups independently selected from: C₁₋₄ alkyl, F, Cl, CORE, and CO₂R^{a}; and,
each R^{a} is independently selected from: H and C₁₋₂ alkyl.

In another aspect, the compound is of Formula I or a stereoisomer or pharmaceutically acceptable salt thereof, wherein:

ring R⁸ is selected from phenyl, pyridyl, thienyl, furanyl, pyrrolyl, oxazolyl, isoxazolyl, thiazolyl, isothiazolyl, pyrimidyl, and pyridazinyl.

In another aspect, the compound is of Formula I or a stereoisomer or pharmaceutically acceptable salt thereof, wherein:

ring R⁸ is selected from phenyl, thienyl, and pyridyl.

In another aspect, the compound is selected from compounds 1-64 of Table 1:

TABLE 1

| | |
|---|---|
| 1. | 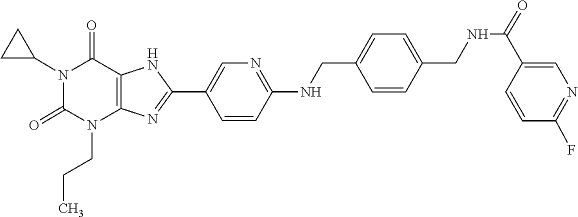 [SMILES: CCCn1c(=O)n(C2CC2)c(=O)c2[nH]c(-c3ccc(NCc4ccc(CNC(=O)c5ccc(F)nc5)cc4)nc3)nc21] |
| 2. | 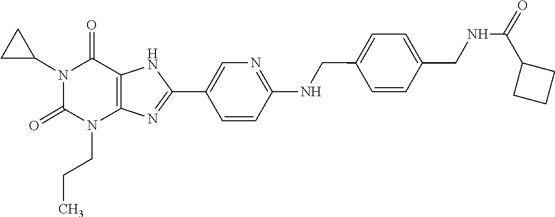 [SMILES: CCCn1c(=O)n(C2CC2)c(=O)c2[nH]c(-c3ccc(NCc4ccc(CNC(=O)C5CCC5)cc4)nc3)nc21] |
| 3. | 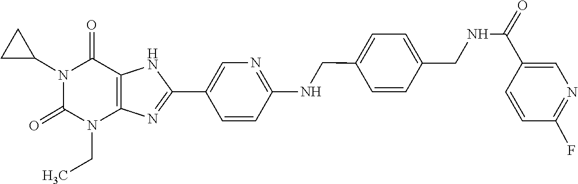 [SMILES: CCn1c(=O)n(C2CC2)c(=O)c2[nH]c(-c3ccc(NCc4ccc(CNC(=O)c5ccc(F)nc5)cc4)nc3)nc21] |
| 4. | 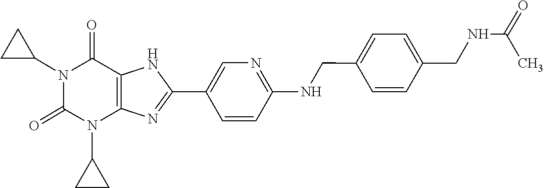 [SMILES: CC(=O)NCc1ccc(CNc2ccc(-c3nc4c([nH]3)c(=O)n(C3CC3)c(=O)n4C3CC3)cn2)cc1] |
| 5. | 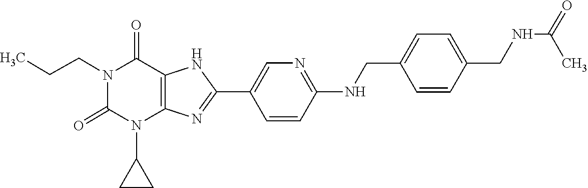 [SMILES: CCCn1c(=O)c2[nH]c(-c3ccc(NCc4ccc(CNC(C)=O)cc4)nc3)nc2n(C2CC2)c1=O] |
| 6. | 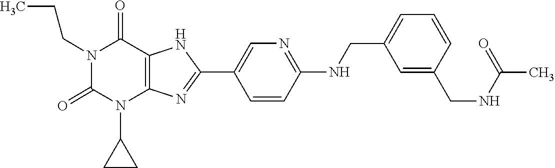 [SMILES: CCCn1c(=O)c2[nH]c(-c3ccc(NCc4cccc(CNC(C)=O)c4)nc3)nc2n(C2CC2)c1=O] |
| 7. | 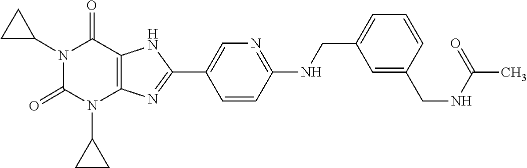 [SMILES: CC(=O)NCc1cccc(CNc2ccc(-c3nc4c([nH]3)c(=O)n(C3CC3)c(=O)n4C3CC3)cn2)c1] |
| 8. | 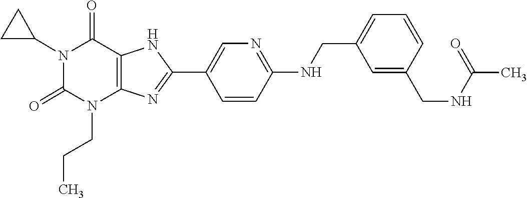 [SMILES: CCCn1c(=O)n(C2CC2)c(=O)c2[nH]c(-c3ccc(NCc4cccc(CNC(C)=O)c4)nc3)nc21] |
| 9. | 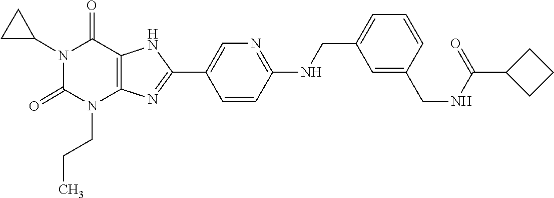 [SMILES: CCCn1c(=O)n(C2CC2)c(=O)c2[nH]c(-c3ccc(NCc4cccc(CNC(=O)C5CCC5)c4)nc3)nc21] |
| 10. | 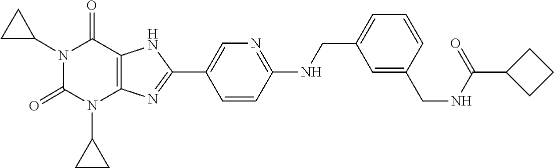 [SMILES: O=C(NCc1cccc(CNc2ccc(-c3nc4c([nH]3)c(=O)n(C3CC3)c(=O)n4C3CC3)cn2)c1)C1CCC1] |
| 11. | 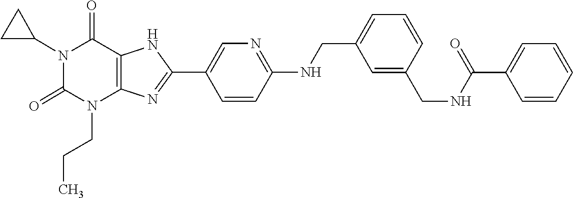 [SMILES: CCCn1c(=O)n(C2CC2)c(=O)c2[nH]c(-c3ccc(NCc4cccc(CNC(=O)c5ccccc5)c4)nc3)nc21] |
| 12. | 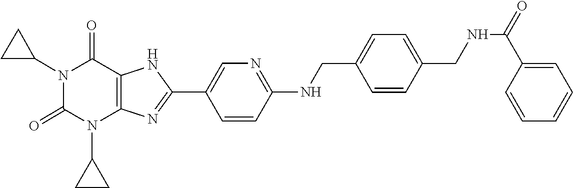 [SMILES: O=C(NCc1ccc(CNc2ccc(-c3nc4c([nH]3)c(=O)n(C3CC3)c(=O)n4C3CC3)cn2)cc1)c1ccccc1] |
| 13. | 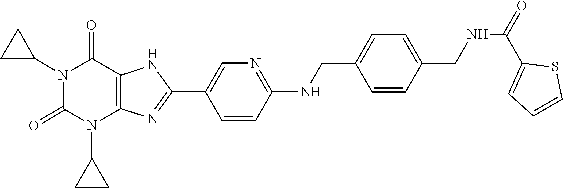 [SMILES: O=C(NCc1ccc(CNc2ccc(-c3nc4c([nH]3)c(=O)n(C3CC3)c(=O)n4C3CC3)cn2)cc1)c1cccs1] |
| 14. | 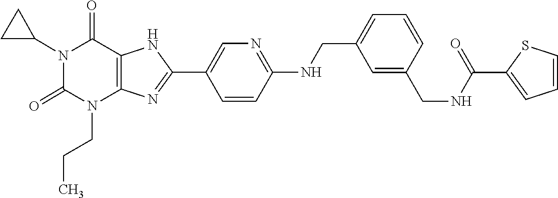 [SMILES: CCCn1c(=O)n(C2CC2)c(=O)c2[nH]c(-c3ccc(NCc4cccc(CNC(=O)c5cccs5)c4)nc3)nc21] |
| 15. | 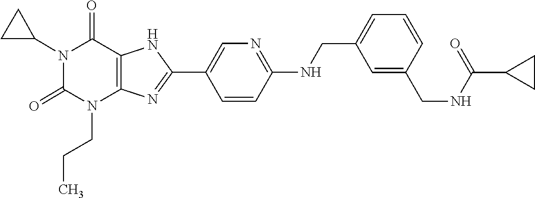 [SMILES: CCCn1c(=O)n(C2CC2)c(=O)c2[nH]c(-c3ccc(NCc4cccc(CNC(=O)C5CC5)c4)nc3)nc21] |
| 16. | 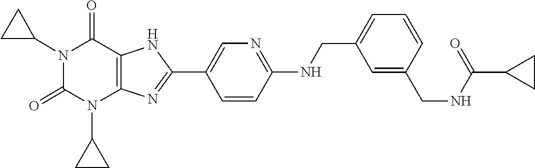 [SMILES: O=C(NCc1cccc(CNc2ccc(-c3nc4c([nH]3)c(=O)n(C3CC3)c(=O)n4C3CC3)cn2)c1)C1CC1] |
| 17. | 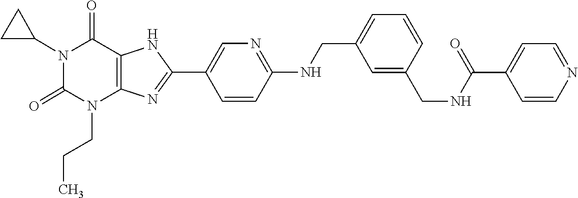 [SMILES: CCCn1c(=O)n(C2CC2)c(=O)c2[nH]c(-c3ccc(NCc4cccc(CNC(=O)c5ccncc5)c4)nc3)nc21] |
| 18. | 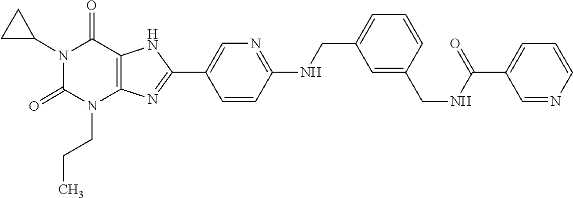 [SMILES: CCCn1c(=O)n(C2CC2)c(=O)c2[nH]c(-c3ccc(NCc4cccc(CNC(=O)c5cccnc5)c4)nc3)nc21] |
| 19. | 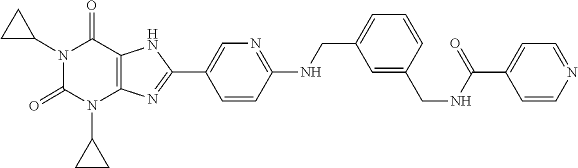 [SMILES: O=C(NCc1cccc(CNc2ccc(-c3nc4c([nH]3)c(=O)n(C3CC3)c(=O)n4C3CC3)cn2)c1)c1ccncc1] |
| 20. | 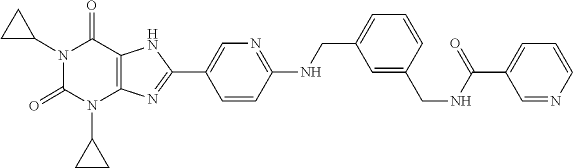 [SMILES: O=C(NCc1cccc(CNc2ccc(-c3nc4c([nH]3)c(=O)n(C3CC3)c(=O)n4C3CC3)cn2)c1)c1cccnc1] |
| 21. | 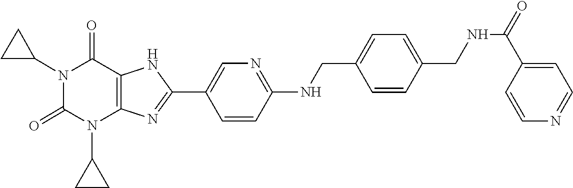 [SMILES: O=C(NCc1ccc(CNc2ccc(-c3nc4c([nH]3)c(=O)n(C3CC3)c(=O)n4C3CC3)cn2)cc1)c1ccncc1] |
| 22. | 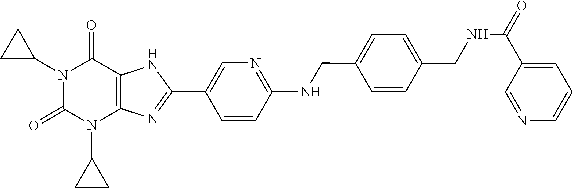 [SMILES: O=C(NCc1ccc(CNc2ccc(-c3nc4c([nH]3)c(=O)n(C3CC3)c(=O)n4C3CC3)cn2)cc1)c1cccnc1] |
| 23. | 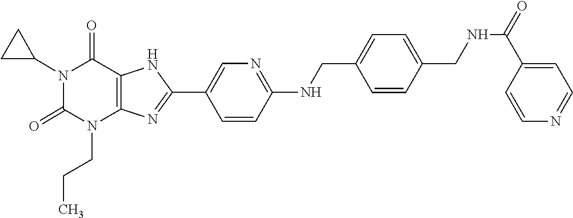 [SMILES: CCCn1c(=O)n(C2CC2)c(=O)c2[nH]c(-c3ccc(NCc4ccc(CNC(=O)c5ccncc5)cc4)nc3)nc21] |
| 24. | 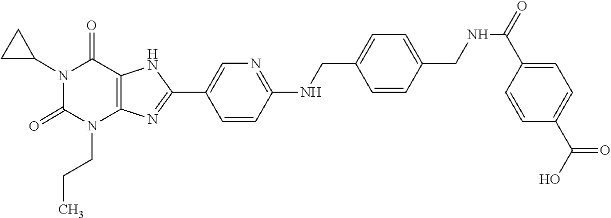 [SMILES: CCCn1c(=O)n(C2CC2)c(=O)c2[nH]c(-c3ccc(NCc4ccc(CNC(=O)c5ccc(C(=O)O)cc5)cc4)nc3)nc21] |
| 25. | 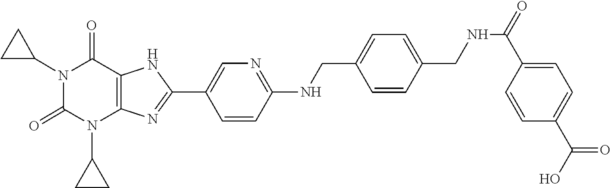 [SMILES: O=C(O)c1ccc(C(=O)NCc2ccc(CNc3ccc(-c4nc5c([nH]4)c(=O)n(C4CC4)c(=O)n5C4CC4)cn3)cc2)cc1] |
| 26. | 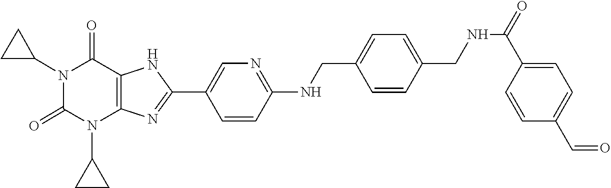 [SMILES: O=Cc1ccc(C(=O)NCc2ccc(CNc3ccc(-c4nc5c([nH]4)c(=O)n(C4CC4)c(=O)n5C4CC4)cn3)cc2)cc1] |
| 27. | 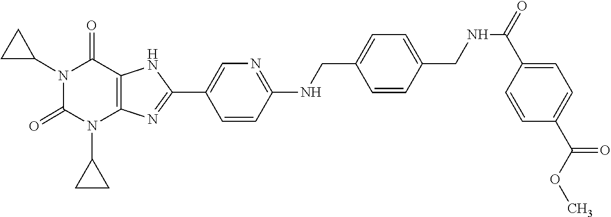 [SMILES: COC(=O)c1ccc(C(=O)NCc2ccc(CNc3ccc(-c4nc5c([nH]4)c(=O)n(C4CC4)c(=O)n5C4CC4)cn3)cc2)cc1] |
| 28. | 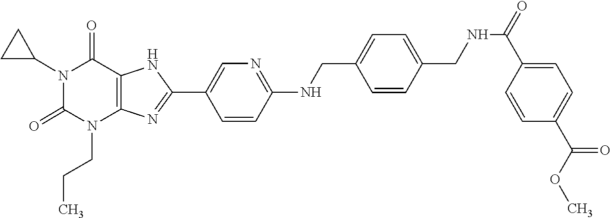 [SMILES: CCCn1c(=O)n(C2CC2)c(=O)c2[nH]c(-c3ccc(NCc4ccc(CNC(=O)c5ccc(C(=O)OC)cc5)cc4)nc3)nc21] |
| 29. | 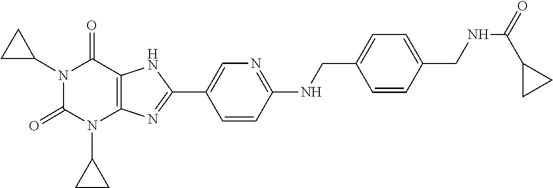 [SMILES: O=C(NCc1ccc(CNc2ccc(-c3nc4c([nH]3)c(=O)n(C3CC3)c(=O)n4C3CC3)cn2)cc1)C1CC1] |
| 30. | 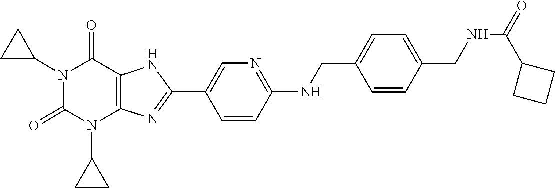 [SMILES: O=C(NCc1ccc(CNc2ccc(-c3nc4c([nH]3)c(=O)n(C3CC3)c(=O)n4C3CC3)cn2)cc1)C1CCC1] |
| 31. | 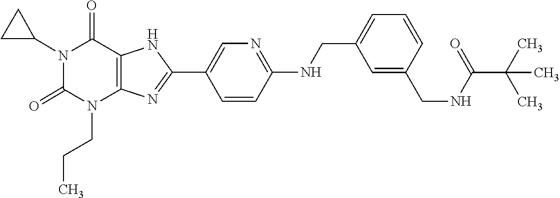 [SMILES: CCCn1c(=O)n(C2CC2)c(=O)c2[nH]c(-c3ccc(NCc4cccc(CNC(=O)C(C)(C)C)c4)nc3)nc21] |
| 32. | 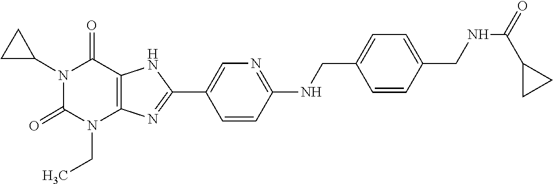 [SMILES: CCn1c(=O)n(C2CC2)c(=O)c2[nH]c(-c3ccc(NCc4ccc(CNC(=O)C5CC5)cc4)nc3)nc21] |
| 33. | 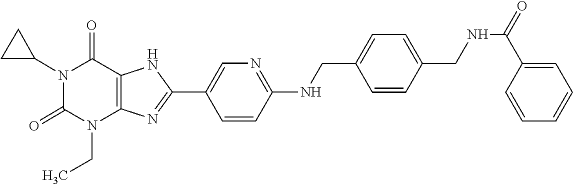 [SMILES: CCn1c(=O)n(C2CC2)c(=O)c2[nH]c(-c3ccc(NCc4ccc(CNC(=O)c5ccccc5)cc4)nc3)nc21] |
| 34. | 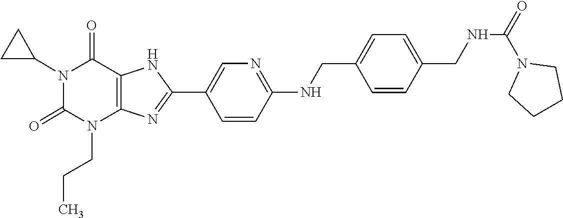 [SMILES: CCCn1c(=O)n(C2CC2)c(=O)c2[nH]c(-c3ccc(NCc4ccc(CNC(=O)N5CCCC5)cc4)nc3)nc21] |
| 35. | 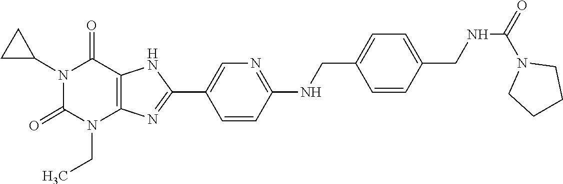 [SMILES: CCn1c(=O)n(C2CC2)c(=O)c2[nH]c(-c3ccc(NCc4ccc(CNC(=O)N5CCCC5)cc4)nc3)nc21] |
| 36. | 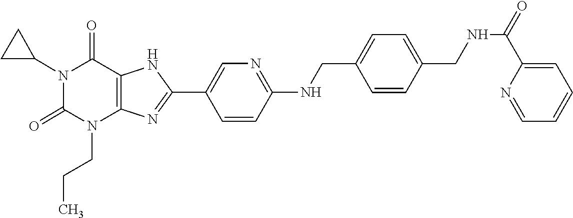 [SMILES: CCCn1c(=O)n(C2CC2)c(=O)c2[nH]c(-c3ccc(NCc4ccc(CNC(=O)c5ccccn5)cc4)nc3)nc21] |
| 37. | 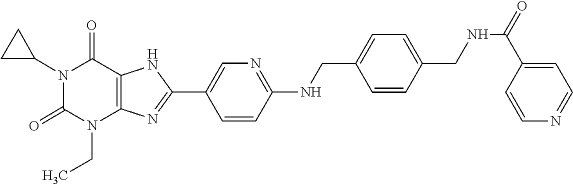 [SMILES: CCn1c(=O)n(C2CC2)c(=O)c2[nH]c(-c3ccc(NCc4ccc(CNC(=O)c5ccncc5)cc4)nc3)nc21] |
| 38. | 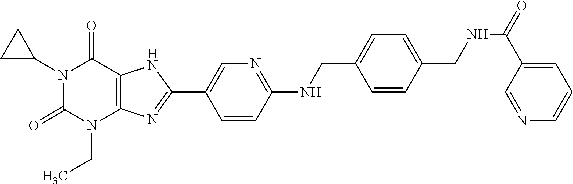 [SMILES: CCn1c(=O)n(C2CC2)c(=O)c2[nH]c(-c3ccc(NCc4ccc(CNC(=O)c5cccnc5)cc4)nc3)nc21] |
| 39. | 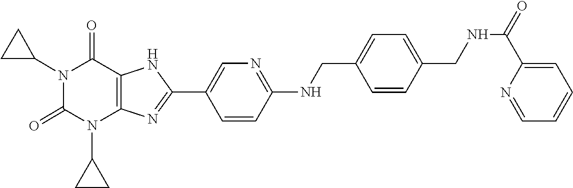 [SMILES: O=C(NCc1ccc(CNc2ccc(-c3nc4c([nH]3)c(=O)n(C3CC3)c(=O)n4C3CC3)cn2)cc1)c1ccccn1] |
| 40. | 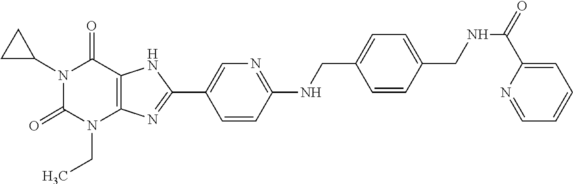 [SMILES: CCn1c(=O)n(C2CC2)c(=O)c2[nH]c(-c3ccc(NCc4ccc(CNC(=O)c5ccccn5)cc4)nc3)nc21] |
| 41. | 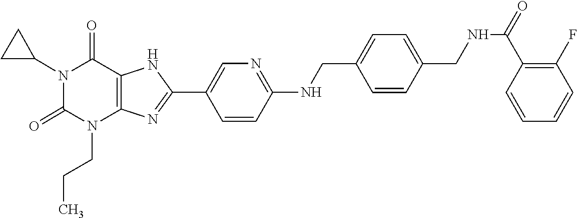 [SMILES: CCCn1c(=O)n(C2CC2)c(=O)c2[nH]c(-c3ccc(NCc4ccc(CNC(=O)c5ccccc5F)cc4)nc3)nc21] |
| 42. | 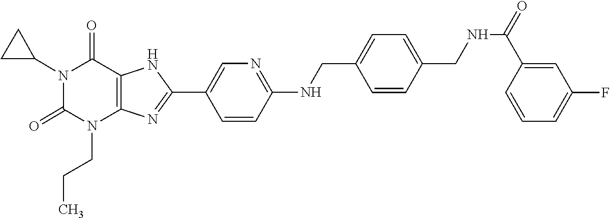 [SMILES: CCCn1c(=O)n(C2CC2)c(=O)c2[nH]c(-c3ccc(NCc4ccc(CNC(=O)c5cccc(F)c5)cc4)nc3)nc21] |
| 43. | 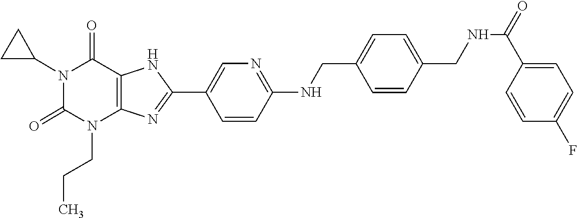 [SMILES: CCCn1c(=O)n(C2CC2)c(=O)c2[nH]c(-c3ccc(NCc4ccc(CNC(=O)c5ccc(F)cc5)cc4)nc3)nc21] |
| 44. | 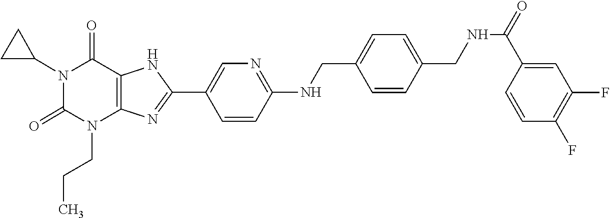 [SMILES: CCCn1c(=O)n(C2CC2)c(=O)c2[nH]c(-c3ccc(NCc4ccc(CNC(=O)c5ccc(F)c(F)c5)cc4)nc3)nc21] |
| 45. | 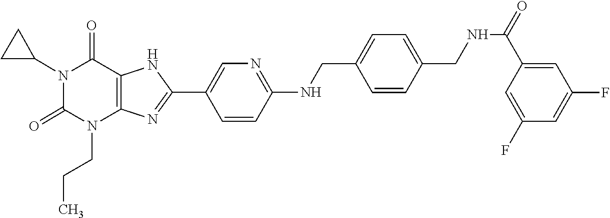 [SMILES: CCCn1c(=O)n(C2CC2)c(=O)c2[nH]c(-c3ccc(NCc4ccc(CNC(=O)c5cc(F)cc(F)c5)cc4)nc3)nc21] |
| 46. | 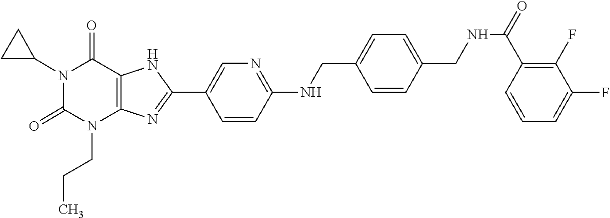 [SMILES: CCCn1c(=O)n(C2CC2)c(=O)c2[nH]c(-c3ccc(NCc4ccc(CNC(=O)c5cccc(F)c5F)cc4)nc3)nc21] |
| 47. | 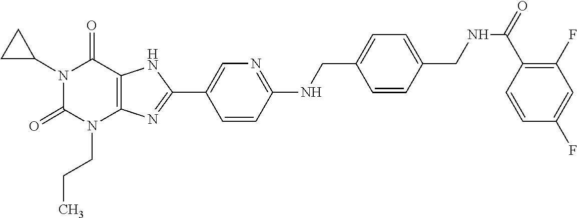 [SMILES: CCCn1c(=O)n(C2CC2)c(=O)c2[nH]c(-c3ccc(NCc4ccc(CNC(=O)c5ccc(F)cc5F)cc4)nc3)nc21] |
| 48. | 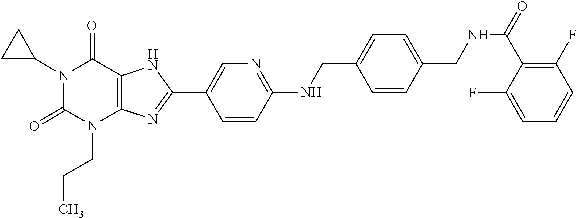 [SMILES: CCCn1c(=O)n(C2CC2)c(=O)c2[nH]c(-c3ccc(NCc4ccc(CNC(=O)c5c(F)cccc5F)cc4)nc3)nc21] |
| 49. | 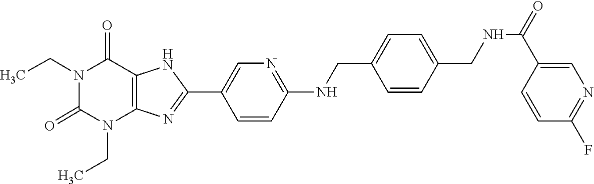 [SMILES: CCn1c(=O)c2[nH]c(-c3ccc(NCc4ccc(CNC(=O)c5ccc(F)nc5)cc4)nc3)nc2n(CC)c1=O] |
| 50. | 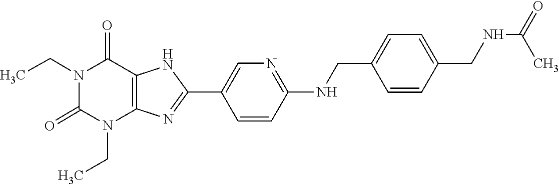 [SMILES: CCn1c(=O)c2[nH]c(-c3ccc(NCc4ccc(CNC(C)=O)cc4)nc3)nc2n(CC)c1=O] |
| 51. | 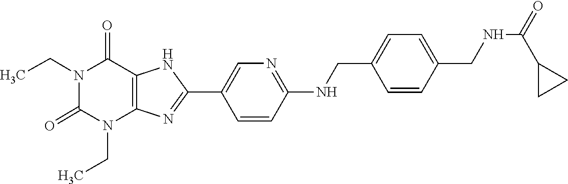 [SMILES: CCn1c(=O)c2[nH]c(-c3ccc(NCc4ccc(CNC(=O)C5CC5)cc4)nc3)nc2n(CC)c1=O] |
| 52. | 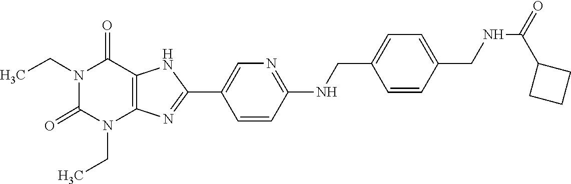 [SMILES: CCn1c(=O)c2[nH]c(-c3ccc(NCc4ccc(CNC(=O)C5CCC5)cc4)nc3)nc2n(CC)c1=O] |
| 53. | 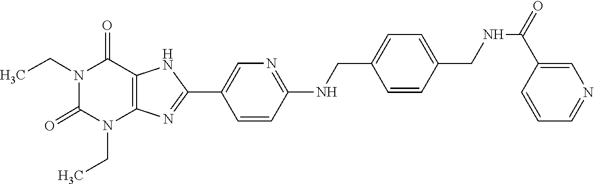 [SMILES: CCn1c(=O)c2[nH]c(-c3ccc(NCc4ccc(CNC(=O)c5cccnc5)cc4)nc3)nc2n(CC)c1=O] |
| 54. | 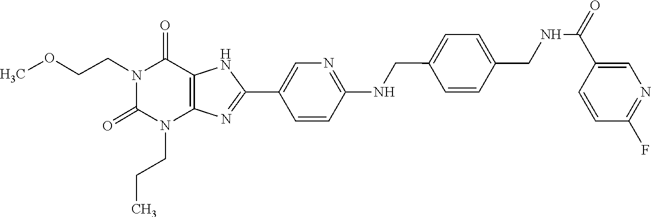 [SMILES: CCCn1c(=O)n(CCOC)c(=O)c2[nH]c(-c3ccc(NCc4ccc(CNC(=O)c5ccc(F)nc5)cc4)nc3)nc21] |
| 55. | 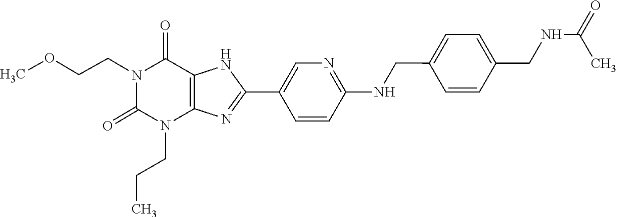 [SMILES: CCCn1c(=O)n(CCOC)c(=O)c2[nH]c(-c3ccc(NCc4ccc(CNC(C)=O)cc4)nc3)nc21] |
| 56. | 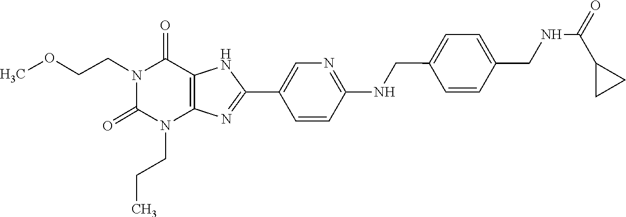 [SMILES: CCCn1c(=O)n(CCOC)c(=O)c2[nH]c(-c3ccc(NCc4ccc(CNC(=O)C5CC5)cc4)nc3)nc21] |
| 57. | 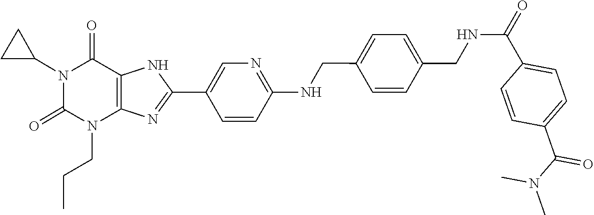 [SMILES: CCCn1c(=O)n(C2CC2)c(=O)c2[nH]c(-c3ccc(NCc4ccc(CNC(=O)c5ccc(C(=O)N(C)C)cc5)cc4)nc3)nc21] |
| 58. | 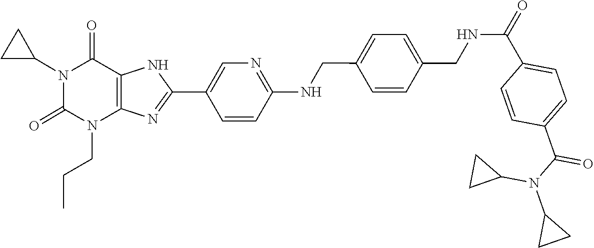 [SMILES: CCCn1c(=O)n(C2CC2)c(=O)c2[nH]c(-c3ccc(NCc4ccc(CNC(=O)c5ccc(C(=O)N(C6CC6)C6CC6)cc5)cc4)nc3)nc21] |
| 59. | 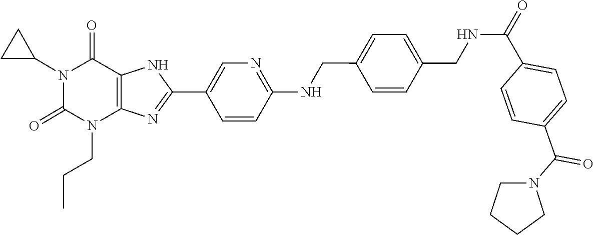 [SMILES: CCCn1c(=O)n(C2CC2)c(=O)c2[nH]c(-c3ccc(NCc4ccc(CNC(=O)c5ccc(C(=O)N6CCCC6)cc5)cc4)nc3)nc21] |
| 60. | 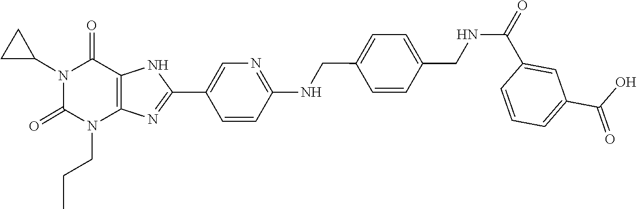 [SMILES: CCCn1c(=O)n(C2CC2)c(=O)c2[nH]c(-c3ccc(NCc4ccc(CNC(=O)c5cccc(C(=O)O)c5)cc4)nc3)nc21] |
| 61. | 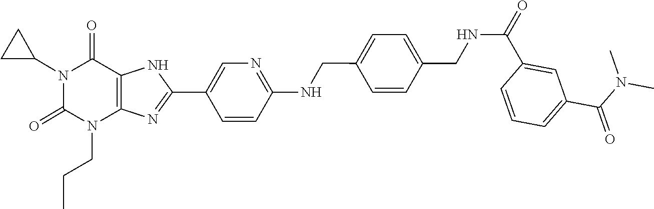 [SMILES: CCCn1c(=O)n(C2CC2)c(=O)c2[nH]c(-c3ccc(NCc4ccc(CNC(=O)c5cccc(C(=O)N(C)C)c5)cc4)nc3)nc21] |
| 62. | 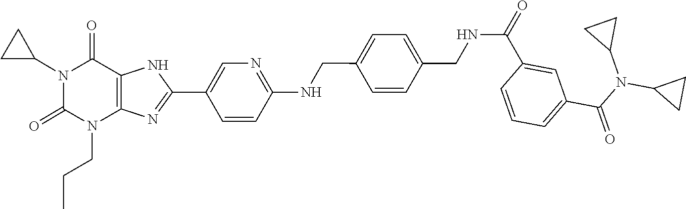 [SMILES: CCCn1c(=O)n(C2CC2)c(=O)c2[nH]c(-c3ccc(NCc4ccc(CNC(=O)c5cccc(C(=O)N(C6CC6)C6CC6)c5)cc4)nc3)nc21] |
| 63. | 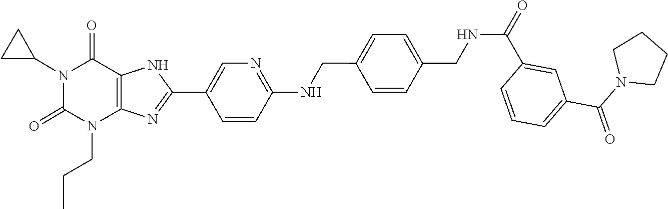 [SMILES: CCCn1c(=O)n(C2CC2)c(=O)c2[nH]c(-c3ccc(NCc4ccc(CNC(=O)c5cccc(C(=O)N6CCCC6)c5)cc4)nc3)nc21] |
| 64. | 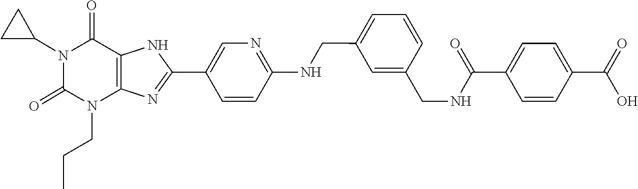 [SMILES: CCCn1c(=O)n(C2CC2)c(=O)c2[nH]c(-c3ccc(NCc4cccc(CNC(=O)c5ccc(C(=O)O)cc5)c4)nc3)nc21] |

or a pharmaceutically acceptable salt thereof.

In another aspect, the present invention provides a novel compound of Formula I, or a stereoisomer or pharmaceutically acceptable salt thereof, wherein: one or more H are replaced by D. For example, R¹ can be a deuterated methyl group (e.g., CD₃) or the alkynyl hydrogens can be replaced by deuterium (—CD₂—). In addition, the groups recited in R¹, R², R³, R⁴, R⁵, R⁶, R⁷, R⁸, R^{a}, and R^{b} that contain a hydrogen (e.g., alkyl, cycloalkyl, alkylene, aryl, and heteroaryl) can be partially or fully replaced by D (e.g., CD₃, CD₂CD₃, CD₂CD(CD₃)₂, d₅-cyclopropyl, d₇-cyclobutyl, d₉-cyclopentyl, d₅-cyclopropyl-CD₂, d₅-phenyl, d₄-phenyl (one substituent is present), d₃-phenyl (two substituents are present), d₄-pyridyl, d₃-pyridyl (one substituent is present), and d₂-pyridyl (two substituent are present).

Deuterium-enriched compounds of the present invention can be prepared by a number of known methods including deuterium exchange of acid labile hydrogens (e.g., contacting the compound with NaOD in D₂O) and using deuterated starting materials (e.g., deuterated iodo-adenosine-uronamide.

In another aspect, the present invention provides a novel pharmaceutical composition, comprising: a therapeutically effective amount of a compound of the present invention or a stereoisomer or pharmaceutically acceptable salt thereof and a pharmaceutically acceptable carrier.

In another aspect, the present invention provides methods of treating a pathological condition or symptom in a mammal for which the A_{2A}-A_{2B} receptors are implicated and antagonism of the receptors provides therapeutic benefit by administering to a subject an effective amount of a compound of the present invention.

In another aspect, the present invention provides a method for treating an adenosine A_{2A}-A_{2B} receptor associated state in a subject, comprising: administering to the subject therapeutically effective amount of a compound of the present invention or a stereoisomer or pharmaceutically acceptable salt thereof.

In another aspect, the adenosine A_{2A}-A_{2B} receptor associated state is cancer. Examples of cancer include: bladder, breast, colorectal, lung, melanoma, prostate, pancreatic, kidney, gastric, and leukemia.

In another aspect, the adenosine A_{2A}-A_{2B} receptor associated state is is an adenosine A_{2B} receptor associated state. In another aspect, the adenosine A_{2B} receptor associated state is selected from: asthma, insulin resistance, atherosclerosis, and fatty liver disease.

In another aspect, the present invention provides a compound for use in therapy.

In another aspect, the present invention provides the use of compounds for the manufacture of a medicament for the treatment of an indication recited herein.

In another aspect, examples of the molecular weight of the compounds of the present invention include (a) less than about 500, 550, 600, 650, 700, 750, 800, 850, 900, 950, or 1000 grams per mole; (b) less than about 950 grams per mole; (c) less than about 850 grams per mole; and, (d) less than about 750 grams per mole.

In another aspect, examples of the solubility of the compounds of the present invention include greater than 50 60, 70, 80, 90, 100, 110, 120, 130, 140, 150, 160, 170, 180, 190, 200, 210, 220, 230, 240, 250, 260, 270, 280, 290, 300, 400, 500, 600, 700, 800, 900 and 1000 μg/mL.

The present invention may be embodied in other specific forms without departing from the spirit or essential attributes thereof. This invention encompasses all combinations of aspects of the invention noted herein. It is understood that any and all embodiments of the present invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

Definitions

The examples provided in the definitions present in this application are non-inclusive unless otherwise stated. They include but are not limited to the recited examples.

A compound or compounds of the present invention, as used herein, includes, where appropriate, stereoisomers and/or pharmaceutically acceptable salts thereof.

“Adenosine A_{2A}-A_{2B} receptor antagonists” include compounds that deactivate the adenosine A_{2A} and/or A_{2B} receptor with a Kᵢ of <1 μM as determined by a known binding assay. An adenosine A_{2A} and/or A_{2B} receptor antagonist may also be cross reactive with other adenosine receptor subtypes (e.g., A₁ and A₃). In one embodiment, the adenosine A_{2A} and/or A_{2B} receptor antagonist may be selective for A_{2A} and/or A_{2B} (e.g., at least 2, 10, 50, or 100/l over another adenosine receptor subtype) or may activate/antagonize other receptors with a greater or lesser affinity than the A_{2A} and/or A_{2B} receptor.

“Adenosine A_{2A} and/or A_{2B} receptor associated state” includes those diseases or disorders which are directly or indirectly implicated in the adenosine A_{2A} and/or A_{2B} receptor pathway. Without being bound by theory, it is thought that administration of an adenosine A_{2A} and/or A_{2B} antagonist blocks the biological activity of natural adenosine at the A_{2A} and/or A_{2B} receptor. Accordingly, an adenosine A_{2A} and/or A_{2B} receptor associated state includes those diseases and disorders directly associated with the activity of the adenosine A_{2A} and/or A_{2B} receptor or the activity of the biological pathway associated with the adenosine A_{2A} and/or A_{2B} receptor.

The compounds herein described may have asymmetric centers, geometric centers (e.g., double bond), or both. All chiral, diastereomeric, racemic forms and all geometric isomeric forms of a structure are intended, unless the specific stereochemistry or isomeric form is specifically indicated. Compounds of the present invention containing an asymmetrically substituted atom may be isolated in optically active or racemic forms. It is well known in the art how to prepare optically active forms, such as by resolution of racemic forms, by synthesis from optically active starting materials, or through use of chiral auxiliaries. Geometric isomers of olefins, C═N double bonds, or other types of double bonds may be present in the compounds described herein, and all such stable isomers are included in the present invention. Specifically, cis and trans geometric isomers of the compounds of the present invention may also exist and may be isolated as a mixture of isomers or as separated isomeric forms. All processes used to prepare compounds of the present invention and intermediates made therein are considered to be part of the present invention. All tautomers of shown or described compounds are also considered to be part of the present invention.

The present invention includes all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The term “substituted” means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties.

“Stable” means that the compound is suitable for pharmaceutical use.

The present invention covers stable compounds and thus avoids, unless otherwise specified, the following bond types: heteroatom-halogen, N—S, O—S, O—O, and S—S.

“Alkyl” includes both branched and straight-chain saturated aliphatic hydrocarbon groups having the specified number of carbon atoms. C₁₋₆ alkyl, for example, includes C₁, C₂, C₃, C₄, C₅, and C₆ alkyl groups. Examples of alkyl include methyl, ethyl, n-propyl, i-propyl, n-butyl, s-butyl, t-butyl, n-pentyl, and s-pentyl.

When an “ene” terminates a group it indicates the group is attached to two other groups. For example, methylene refers to a —CH₂-moiety.

“Alkenyl” includes the specified number of hydrocarbon atoms in either straight or branched configuration with one or more unsaturated carbon-carbon bonds that may occur in any stable point along the chain, such as ethenyl and propenyl. C₂₋₆alkenyl includes C₂, C₃, C₄, C₅, and C₆alkenyl groups.

“Alkynyl” includes the specified number of hydrocarbon atoms in either straight or branched configuration with one or more triple carbon-carbon bonds that may occur in any stable point along the chain, such as ethynyl and propynyl. C₂₋₆Alkynyl includes C₂, C₃, C₄, C₅, and C₆alkynyl groups.

“Alkoxy” includes alkyl, alkenyl, and alkynyl groups covalently linked to an oxygen atom. Examples of alkoxy groups include methoxy, ethoxy, isopropyloxy, propoxy, t-butyloxy, isobutyloxy, butoxy, and pentoxy groups.

“Cycloalkyl” includes the specified number of hydrocarbon atoms in a saturated ring, such as cyclopropyl, cyclobutyl, cyclopentyl, cyclohexyl, cycloheptyl, and cyclooctyl. C₃₋₈ cycloalkyl includes C₃, C₄, C₅, C₆, C₇, and C₈ cycloalkyl groups.

“Halo” or “halogen” refers to fluoro, chloro, bromo, and iodo.

“Aryl” refers to any stable 6, 7, 8, 9, 10, 11, 12, or 13 membered monocyclic, bicyclic, or tricyclic ring, wherein at least one ring, if more than one is present, is aromatic. Examples of aryl include fluorenyl, phenyl, naphthyl, indanyl, adamantyl, and tetrahydronaphthyl.

“Heteroaryl” refers to any stable 5, 6, 7, 8, 9, 10, 11, or 12 membered (unless the number of members is otherwise recited) monocyclic, bicyclic, or tricyclic heterocyclic ring that is aromatic, and which consists of carbon atoms and 1, 2, 3, or 4 heteroatoms independently selected from the group consisting of N, O, and S. If the heteroaryl group is bicyclic or tricyclic, then at least one of the two or three rings must contain a heteroatom, though both or all three may each contain one or more heteroatoms. If the heteroaryl group is bicyclic or tricyclic, then only one of the rings must be aromatic. The N group may be N, NH, or N-substituent, depending on the chosen ring and if substituents are recited. The nitrogen and sulfur heteroatoms may optionally be oxidized (e.g., S, S(O), S(O)₂, and N—O). The heteroaryl ring may be attached to its pendant group at any heteroatom or carbon atom that results in a stable structure. The heteroaryl rings described herein may be substituted on carbon or on a nitrogen atom if the resulting compound is stable.

Examples of heteroaryl includes acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, naphthyridinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, pyranyl, pyrazinyl, pyrazolyl, pyridazinyl, pyridooxazole, pyridoimidazole, pyridothiazole, pyridinyl, pyridyl, pyrimidinyl, 2H-pyrrolyl, pyrrolyl, quinazolinyl, quinolinyl, 4H-quinolizinyl, quinoxalinyl, quinuclidinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

The term “heterocycle” or “heterocyclyl” includes stable 4, 5, 6, 7, 8, 9, 10, 11, or 12 membered (unless the number of members is otherwise recited) monocyclic, bicyclic, or tricyclic heterocyclic ring that is saturated or partially unsaturated, and which consists of carbon atoms and 1, 2, 3, or 4 heteroatoms independently selected from the group consisting of N, O, and S. If the heterocycle is defined by the number of carbon atoms, then from 1, 2, 3, or 4 of the listed carbon atoms are replaced by a heteroatom. If the heterocycle is bicyclic or tricyclic, then at least one of the two or three rings must contain a heteroatom, though both or all three may each contain one or more heteroatoms. The N group may be N, NH, or N-substituent, depending on the chosen ring and if substituents are recited. The nitrogen and sulfur heteroatoms optionally may be oxidized (e.g., S, S(O), S(O)₂, and N—O). The heterocycle may be attached to its pendant group at any heteroatom or carbon atom that results in a stable structure. The heterocycles described herein may be substituted on carbon or on a nitrogen atom if the resulting compound is stable.

Examples of heterocycles include, but are not limited to, decahydroquinolinyl, imidazolidinyl, imidazolinyl, indolinyl, isatinoyl, methylenedioxyphenyl, morpholinyl, octahydroisoquinolinyl, oxazolidinyl, oxindolyl, piperazinyl, piperidinyl, piperidonyl, 4-piperidonyl, piperonyl, pyranyl, pyrazolidinyl, pyrazolinyl, pyrrolidinyl, pyrrolinyl, tetrahydrofuranyl, tetrahydroisoquinolinyl, tetrahydroquinolinyl, 1-aza-bicyclo[2.2.2]octane, 2,5-diaza-bicyclo[2.2.2]octane, and 2,5-diaza-bicyclo[2.2.1]heptane. Also included are fused ring and spiro compounds containing, for example, the above heterocycles.

“Mammal” and “patient” cover warm blooded mammals that are typically under medical care (e.g., humans and domesticated animals). Examples include feline, canine, equine, bovine, non-human primate, and human, as well as just human.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting its development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are useful. Lists of suitable salts are found in Remington's Pharmaceutical Sciences, 18th ed., Mack Publishing Company, Easton, Pa., 1990, p 1445, the disclosure of which is hereby incorporated by reference.

“Therapeutically effective amount” includes an amount of a compound of the present invention that is effective when administered alone or in combination to an indication listed herein. “Therapeutically effective amount” also includes an amount of the combination of compounds claimed that is effective to treat the desired indication. The combination of compounds can be a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased effect, or some other beneficial effect of the combination compared with the individual components.

Formulations and Dosages

The compounds of the present invention can be formulated as pharmaceutical compositions and administered to a mammalian host, such as a human patient in a variety of forms adapted to the chosen route of administration, e.g., orally or parenterally, by intravenous (e.g., continuously or bolus), intrathecal, intramuscular, topical, intradermal, intraperitoneal, intraocular, inhalation or subcutaneous routes. Exemplary pharmaceutical compositions are disclosed in “Remington: The Science and Practice of Pharmacy,” A. Gennaro, ed., 20th edition, Lippincott, Williams & Wilkins, Philadelphia, Pa.

Thus, the present compounds may be systemically administered, e.g., orally, in combination with a pharmaceutically acceptable carrier/excipient such as an inert diluent or an assimilable edible carrier. They may be enclosed in hard or soft shell gelatin capsules, may be compressed into tablets or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. The amount of active compound in such therapeutically useful compositions is such that an effective dosage level will be obtained.

The amount of the compound of the present invention or an active salt or derivative thereof, required for use in treatment will vary not only with the particular compound or salt selected but also with the route of administration, the nature of the condition being treated, and the age and condition of the patient, and will ultimately be at the discretion of the attendant physician or clinician. In general, however, a suitable dose will be in the range of (a) about 1.0-1000 mg/kg of body weight per day, (b) about 10-500 mg/kg of body weight per day, and (c) about 5-20 mg/kg of body weight per day.

For an eye drop, the composition will typically contain an active ingredient at a concentration of generally from 0.000001 to 10% (w/v), also from 0.00001 to 3% (w/v), 0.0001 to 1% (w/v), and 0.001 to 0.1% (w/v) may be instilled to an adult once to several times a day.

For oral administration, the compounds of the present invention may be administered to an adult once or divided into several times at a dose of generally from 0.001 to 5000 mg per day, also from 0.1 to 2500 mg per day, and from 1 to 1000 mg per day.

For a liquid composition (e.g., in a lotion), the concentration of compounds of the present invention can be from (a) about 0.1-25 wt % and (b) about 0.5-10 wt %. The concentration in a semi-solid or solid composition such as a gel or a powder can be (a) about 0.1-5 wt % and (b) about 0.5-2.5 wt %.

The compounds of the present invention can be conveniently administered in unit dosage form; e.g., tablets, caplets, etc., containing (a) about 4-400 mg, (b) about 10-200 mg, and (c) about 20-100 mg of active ingredient per unit dosage form.

The compounds of the present invention can be administered to achieve peak plasma concentrations of the active compound of (a) about 0.02-20 μM, (b) about 0.1-10 μM, and (c) about 0.5-5 μM. These concentrations may be achieved, for example, by the intravenous injection (e.g., continuously or bolus) of a 0.005-0.5% solution of the active ingredient, or orally administered as a bolus containing about 4-400 mg of the active ingredient.

When a compound of the present invention is administered in combination with another agent or agents (e.g., co-administered), then the compound of the present invention and other agent can be administered simultaneously or in any order. They can be administered as a single pharmaceutical composition or as separate compositions. The administration of the compound of the present invention can be prior to the other agent(s), within minutes thereof, or up to hours (e.g., 24 or 48) or even days after the administration of the other agent(s). For example, the administration of the compound of the present invention can be within about 24 hours or within about 12 hours.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compounds of the present invention may also be administered intravenously (e.g., continuously or bolus) or intraperitoneally by infusion or injection. Solutions of the compounds of the present invention or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes or miceles, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compounds of the present invention may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings or sprayed onto the affected area using pump-type or aerosol sprayers.

Examples of useful dermatological compositions which can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508). Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The compounds of the present invention can also be administered by inhalation from an inhaler, insufflator, atomizer or pressurized pack or other means of delivering an aerosol spray. Pressurized packs may comprise a suitable propellant such as carbon dioxide or other suitable gas. In case of a pressurized aerosol, the dosage unit may be determined by providing a value to deliver a metered amount. The inhalers, insufflators, and atomizers are fully described in pharmaceutical reference books such as Remington's Pharmaceutical Sciences Volumes 16 (1980) or 18 (1990) Mack Publishing Co.

The desired dose of the compounds of the present invention may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Synthesis

The compounds of the present invention can be prepared in a number of ways known to one skilled in the art of organic synthesis. The compounds of the present invention can be synthesized using the methods described below, together with synthetic methods known in the art of synthetic organic chemistry, or by variations thereon as appreciated by those skilled in the art. Useful methods include, but are not limited to, those described below. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention. It will also be recognized that another major consideration in the planning of any synthetic route in th is field is the judicious choice of the protecting group used for protection of the reactive functional groups present in the compounds described in this invention. An authoritative account describing the many alternatives to the trained practitioner is Greene and Wuts (Protective Groups In Organic Synthesis, Wiley and Sons, 1991). All references cited herein are hereby incorporated in their entirety herein by reference.

One stereoisomer of a compound of the present invention may be a more potent A_{2B} antagonist than its counterpart(s). Thus, stereoisomers are included in the present invention. When required, separation of the racemic material can be achieved by HPLC using a chiral column or by a resolution using a resolving agent such as described in Wilen, S. H. Tables of Resolving Agents and Optical Resolutions 1972, 308 or using enantiomerically pure acids and bases. A chiral compound of the present invention may also be directly synthesized using a chiral catalyst or a chiral ligand, e.g., Jacobsen, E. Acc. Chem. Res. 2000, 33, 421-431 or using other enantio-and diastereo-selective reactions and reagents known to one skilled in the art of asymmetric synthesis.

Other features of the invention will become apparent in the course of the following descriptions of exemplary embodiments that are given for illustration of the invention and are not intended to be limiting thereof.

### EXAMPLES

The following examples are representative of the procedures used to prepare the compounds of the present invention.

The compounds of Formula I can be prepared by a similar methods described in P. J. Scammells, et al., J. Med. Chem. 37, 2704-2712 (1994). For example, 1,3-disubstituted-8-(6-chloropyridin-3-yl)xanthine (1a), which can be prepared as described in Scammells et al., can be reacted with p, m or o-xylylenediamine (2a) at to afford the desired intermediate 3a. Reaction of the intermediate 3a with an appropriate acid chloride can afford the desired final product 4a.

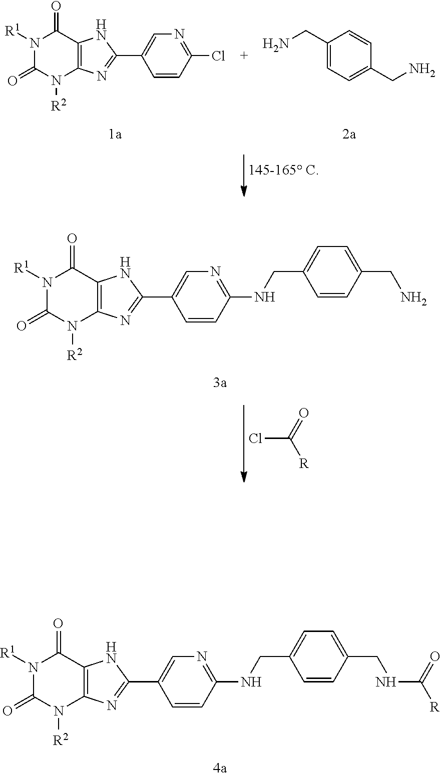

Procedure for the synthesis of 1,3-Dicyclopropyl-8-[6-({[4-(aminomethyl)phenyl]methyl}amino)pyridin-3-yl]xanthine:

1,3-Dicyclopropyl-8-(6-chloropyridin-3-yl)xanthine (0.9529 g, 2.7720 mmoles) and p-xylylenediamine (6.3272 g, 46.4586 mmoles) were combined in a pressure flask with 1-propanol (˜20 mL). The mixture stirred at 165° C. overnight. The solvent was removed in vacuo, and the solid was washed and filtered with IPA to yield 0.4424 g of product.

Procedure for the synthesis of 8-(6-((4-(aminomethyl)benzyl)amino)pyridin-3-yl)-1-cyclopropyl-3-ethylxanthine:

8-(6-Chloropyridin-3-yl)-1-cyclopropyl-3-ethylxanthine (1.0000 g, 3.0142 mmoles) and p-xylylenediamine (6.8801 g, 50.5184 mmoles) were combined in a pressure flask with a small amount of 1-propanol (˜20 mL). The mixture stirred at 165° C. overnight. The solution was filtered with IPA to yield ˜0.5000 g of product.

Procedure for the synthesis of 1-cyclopropyl-3-propyl-8-[6-({[3-(aminomethyl)phenyl]methyl}amino)pyridin-3-yl]xanthine:

1-Cyclopropyl-3-propyl-8-(6-chloropyridin-3-yl)xanthine (1.0000 g, 2.8920 mmoles) was combined with m-xylylenediamine (6.6174 g, 48.5858 mmoles) in pyridine (˜20 mL) and allowed to stir at 165° C. for 24 hours. The solvent was removed in vacuo and the solid was filtered and washed with IPA to yield the product.

Procedure for synthesis of 3-cyclopropyl-1-propyl-8-[6-({[4-(aminomethyl)phenyl]methyl}amino)pyridin-3-yl]xanthine:

8-(6-Chloropyridin-3-yl)-1-propyl-3-cyclopropylxanthine (0.5000 g, 1.4460 mmoles) and p-xylylenediamine (3.3005 g, 24.2344 mmoles) were combined in a pressure flask with a small amount of 1-propanol (˜20 mL). The mixture was stirred at 165° C. overnight. The solution was filtered with IPA to yield the product.

Procedure for the synthesis of 1-cyclopropyl-3-propyl-8-[6-({[4-(aminomethyl)phenyl]methyl}amino)pyridin-3-yl]xanthine:

8-(6-Chloropyridin-3-yl)-1-cyclopropyl-3-propylxanthine (0.8021 g, 2.3197 mmoles) and p-xylylenediamine (5.2948 g, 38.8779 mmoles) were combined in a pressure flask with a small amount of 1-propanol (˜20 mL). The mixture stirred at 165° C. overnight. The solution was filtered with IPA to yield ˜0.5000 g of product.

### EXAMPLE 1

### 1-Cyclopropyl-3-propyl-8-[6-((4-((6-fluoronicotinamido)methyl)benzyl)amino)pyridin-3-yl]xanthine

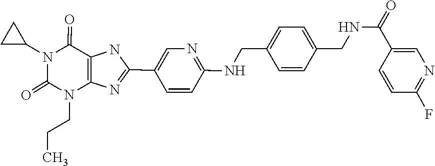

8-(6-((4-(Aminomethyl)benzyl)amino)pyridine-3-yl)-1-cyclopropyl-3-propylxanthine (0.1000 g, 0.2245 mmoles), pyridine (14.46 mL, 179.1510 mmoles) and 6-fluoronicotinoyl chloride (0.0448 g, 0.2806 mmoles). The mixture stirred at 25° C. for 24 hours. The solvent was removed under vacuum. Methanol (˜20 mL) was added to the reaction, which was then filtered and washed with methanol to afford the product, 65.9 mg, 0.1159 mmoles, 51.64% yield. HPLC-MS conditions: 40%-80% MeOH (0.1% formic acid)/H₂O (0.1% formic acid) 10 min. 5 min. hold, Rt=10.091, LRMS ESI (M+H⁺) 569.45.

### EXAMPLE 2

### 1-Cyclopropyl-3-propyl-8-[6-((4-cyclobutylcarboxamido-methyl)benzyl)amino)pyrid-3-yl]xanthine

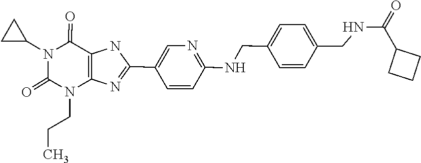

8-(6-((4-(Aminomethyl)benzyl)amino)pyridin-3-yl)-1-cyclopropyl-3-propylxanthine (0.2000 g, 0.4489 mmoles) and cyclobutanecarbonyl chloride (0.0798 g, 0.6734 mmoles) were combined in a flask with dry pyridine (15 mL). The mixture stirred at 25° C. for 24 hours. The solvent was removed in vacuo to yield product. A silica plug was run with 0, 1, 2.5 and 5% DCM/MeOH. The product was isolated and purified by column chromatography. Like fractions were collected, and the solvent was removed in vacuo to give product, 135 mg, 0.2559 mmoles, 57.00% yield. HPLC-MS conditions: 40%-80% MeOH (0.1% formic acid)/H₂O (0.1% formic acid) 10 min. 5 min. hold, Rt=10.179, LRMS ESI (M+H⁺) 528.45.

### EXAMPLE 3

### 1-Cyclopropyl-3-ethyl-8-(6-((4-((6-fluoronicotinamido)methyl)benzyl)amino)pyridin-3-yl)xanthine:

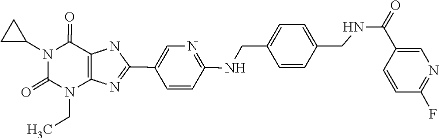

8-(6-((4-(Aminomethyl)benzyl)amino)pyridin-3-yl)-1-cyclopropyl-3-ethylxanthine (0.1500 g, 0.3477 mmoles) was combined with 6-fluoronicotinoyl chloride (0.1498 g, 0.1064 mL, 0.9388 mmoles) in dry pyridine (20 mL) and allowed to stir at 25° C. for 24 hours. The solvent was removed in vacuo and the reaction was filtered and washed with DCM to afford the product, 0.1334 g, 0.2405 mmoles, 69.18% yield. HPLC-MS conditions: 40%-80% MeOH (0.1% formic acid)/H₂O (0.1% formic acid) 10 min. 5 min. hold, Rt=9.049, LRMS ESI (M+H⁺) 555.45.

### EXAMPLE 4

### 1,3-Dicyclopropyl-8-[6-((4-(acetamidomethyl)benzyl)amino)pyrid-3-yl]xanthine:

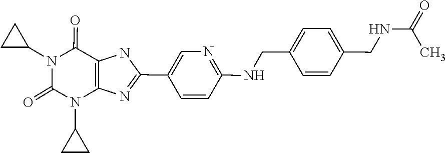

8-(6-((4-(Aminomethyl)benzyl)amino)pyridin-3-yl)-1,3-dicyclopropylxanthine (0.1000 g, 0.2255 mmoles) and acetyl chloride (0.0266 g, 0.3382 mmoles) were combined in a flask with dry pyridine (15 mL). The reaction stirred at 25° C. for 24 hours. The reaction was checked via HPLC for completion, and another 10 uL of acetyl chloride was added. The reaction stirred an additional 24 hours. The solvent was removed in vacuo, and DCM (˜20 mL) was added to the solid. The reaction was filtered and washed with DCM to yield the product, 0.0690 g, 0.1421 mmoles, 63.03% yield. HPLC-MS conditions: 40%-80% MeOH (0.1% formic acid)/H₂O (0.1% formic acid) 10 min. 5 min. hold, Rt: 7.169, LRMS ESI (M+H⁺) 486.35.

### EXAMPLE 5

### 3-Cyclopropyl-1-propyl-8-[6-((4-(acetamidomethyl)benzyl)amino)pyrid-3-yl]xanthine

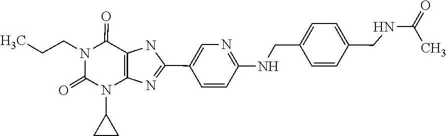

8-(6-((4-(Aminomethyl)benzyl)amino)pyridin-3-yl)-1-propyl-3-cyclopropylxanthine (0.1000 g, 0.2245 mmoles) was combined with acetyl chloride (0.0264 g, 0.3367 mmoles) and dry pyridine (15 mL) in a flask. The mixture stirred at 25° C. for 24 hours. The reaction was checked via HPLC for completion, and another 10 μL of acetyl chloride was added. The reaction stirred an additional 24 hours. The solvent was removed in vacuo, and DCM (˜20 mL) was added. The reaction was filtered and washed with DCM to afford the product, 0.0420 g, 0.0861 mmoles, 38.38% yield. HPLC-MS conditions: 40%-80% MeOH (0.1% formic acid)/H₂O (0.1% formic acid) 10 min. 5 min. hold, Rt=8.677, LRMS ESI (M+H⁺) 488.40.

### EXAMPLE 8

### 1-Cyclopropyl-3-propyl-8-[6-((3-(acetamidomethyl)benzyl)amino)pyrid-3-yl]xanthine:

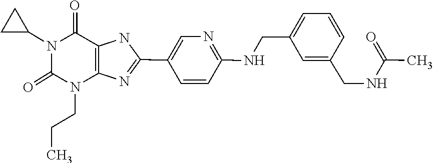

8-(6-((3-(Aminomethyl)benzyl)amino)pyridin-3-yl)-1-cyclopropyl-3-propylxanthine (0.1000 g, 0.2245 mmoles) was combined with acetyl chloride (0.0264 g, 0.3367 mmoles) and dry pyridine (15 mL) in a flask. The mixture stirred at 25° C. for 24 hours. The reaction was checked via HPLC for completion, and another 10 uL of acetyl chloride was added. The reaction stirred an additional 24 hours. The solvent was removed in vacuo and DCM (˜20 mL) was added. The reaction was filtered and washed with DCM to yield the product, 0.0530 g, 0.1087 mmoles, 48.43% yield. HPLC-MS conditions: 40%-80% MeOH (0.1% formic acid)/H₂O (0.1% formic acid) 10 min. 5 min. hold, Rt=9.804, LRMS ESI (M+H⁺) 488.35.

### EXAMPLE 9

### 1-Cyclopropyl-3-propyl-8-(6-((3-(cyclobutylcarboxamidomethyl)benzyl)amino)pyridin-3-yl)xanthine:

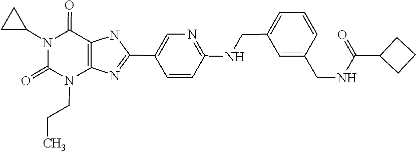

8-(6-((3-(Aminomethyl)benzyl)amino)pyridin-3-yl)-1-cyclopropyl-3-propylxanthine (0.1000 g, 0.2245 mmoles) and cyclobutanecarbonyl chloride (0.0399 g, 0.3367 mmoles) were combined in a flask with dry pyridine (15 mL). The reaction stirred at 25° C. for 24 hours. The reaction was checked for completion via HPLC. More cyclobutanecarbonyl chloride (10 uL) was added and the reaction stirred for 48 hours. The solvent was removed in vacuo and DCM (˜20 mL) was added. The reaction was filtered and washed with DCM to afford the product, 0.0238 g, 0.0451 mmoles, 20.10% yield. HPLC-MS conditions: 40%-80% MeOH (0.1% formic acid)/H₂O (0.1% formic acid) 10 min. 5 min. hold, Rt=8.896, LRMS ESI (M+H⁺) 554.35.

### EXAMPLE 11

### 1-Cyclopropyl-3-propyl-8-(6-((3-(benzamidomethyl)benzyl)amino)pyrid-3-yl)xanthine:

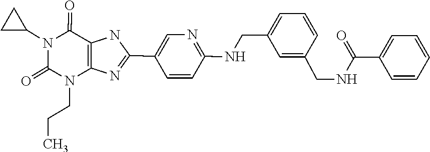

8-(6-((3-(Aminomethyl)benzyl)amino)pyridin-3-yl)-1-cyclopropyl propylxanthine (0.1000 g, 0.2245 mmoles) was combined with benzoyl chloride (0.0473 g, 0.3367 mmoles) and dry pyridine (15 mL) in a flask. The mixture stirred at 25° C. for 24 hours. The reaction was checked via HPLC for completion, and another 10 uL of benzoyl chloride was added. The reaction stirred an additional 24 hours. The solvent was removed in vacuo and DCM (˜20 mL) was added. The reaction was filtered and washed with DCM to yield the product, 0.0630 g, 0.1146 mmoles, 51.07% yield. HPLC-MS conditions: 40%-80% MeOH (0.1% formic acid)/H₂O (0.1% formic acid) 10 min. 5 min. hold, Rt=7.964, LRMS ESI (M+H⁺) 512.40.

### EXAMPLE 13

### 1,3-Dicyclopropyl -8-(6-((4-((thiophenyl-2-carboxamido)methyl)benzyl)amino)pyridin-3-yl)xanthine:

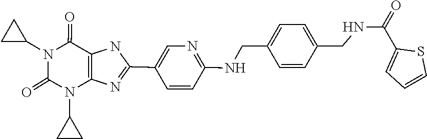

8-[6-({[4-(Aminomethyl)phenyl]methyl}amino)pyridin-3-yl]-1,3-dicyclopropylxanthine (0.1000 g, 0.2255 mmoles) was combined with 2-thiophenecarbonyl chloride (0.0364 g, 0.2480 mmoles) in dry pyridine (15 mL). The reaction was stirred at 25° C. for 24 hours. The solvent was removed in vacuo and methanol (˜20 mL) was added. The reaction was filtered and washed with methanol to afford the product, 0.0670 g, 0.1210 mmoles, 53.67% yield. HPLC-MS conditions: 40%-80% MeOH (0.1% formic acid)/H₂O (0.1% formic acid) 10 min. 5 min. hold, Rt=8.770, LRMS ESI (M+H⁺) 526.40

### EXAMPLE 15

### 1-Cyclopropyl-3-propyl-8-(6-((3-((cyclopropylcarboxamido)methyl)benzyl)amino)pyridin-3-yl)xanthine:

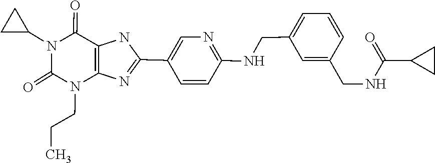

8-(6-((3-(Aminomethyl)benzyl)amino)pyridin-3-yl)-1-cyclopropyl-3-propylxanthine (0.1000 g, 0.2245 mmoles) and cyclopropanecarbonyl chloride (0.0352 g, 0.3367 mmoles) were combined in a flask with dry pyridine (15 mL). The reaction stirred at 25° C. for 24 hours. The reaction was checked for completion via HPLC. More cyclopropanecarbonyl chloride (10 uL) was added and the reaction stirred for 48 hours. The solvent was removed in vacuo and DCM (˜20 mL) was added. The reaction was filtered and washed with DCM to afford the product, 0.0700 g, 0.1363 mmoles, 60.72% yield. HPLC-MS conditions: 40%-80% MeOH (0.1% formic acid)/H₂O (0.1% formic acid) 10 min. 5 min. hold, Rt=10.364, LRMS ESI (M+H⁺) 528.45.

### EXAMPLE 29

### 1,3-Dicyclopropyl-8-[6-((4-(cyclopropylcarboxamidomethyl)benzyl)amino)pyrid-3-yl]xanthine:

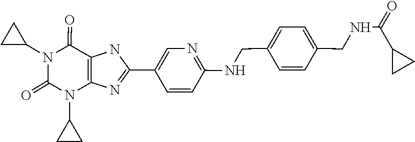

8-(6-((4-(Aminomethyl)benzyl)amino)pyridin-3-yl)-1,3-dicyclopropylxanthine (0.1000 g, 0.2255 mmoles) was combined with cyclopropanecarbonyl chloride (0.0259 g, 0.0225 mmoles) and dry pyridine (15 mL) in a flask. The mixture stirred at 25° C. for 24 hours. The reaction was checked via HPLC for completion, and another 10 uL of cyclopropanecarbonyl chloride was added. The reaction stirred an additional 24 hours. The solvent was removed in vacuo and DCM (˜20 mL) was added. The reaction was filtered and washed with DCM to yield the product, 0.0530 g, 0.1036 mmoles, 45.95% yield. HPLC-MS conditions: 40%-80% MeOH (0.1% formic acid)/H₂O (0.1% formic acid) 10 min. 5 min. hold, Rt=7.964, LRMS ESI (M+H⁺) 512.40.

### EXAMPLE 30

### 1,3-Dicyclopropyl-8-[6-((4-((cyclobutylcarboxamido)methyl)benzyl)amino)pyrid-3-yl]xanthine:

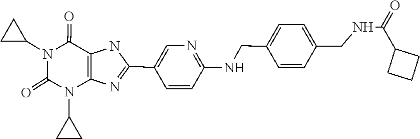

8-[6-({[4-(Aminomethyl)phenyl]methyl}amino)pyridin-3-yl]-1,3-dicyclopropylxanthine (0.1000 g, 0.2255 mmoles) was combined with cyclobutanecarbonyl chloride (0.0294 g, 0.2480 mmoles) in dry pyridine (15 mL). The reaction was stirred at 25° C. for 24 hours. The solvent was removed in vacuo and DCM (˜20 mL) was added. The reaction was filtered and washed with DCM to afford the product, 0.0240 g, 0.0457 mmoles, 20.25% yield. HPLC-MS conditions: 40%-80% MeOH (0.1% formic acid)/H₂O (0.1% formic acid) 10 min. 5 min. hold, Rt=8.770, LRMS ESI (M+H⁺) 526.40.

### EXAMPLE 31

### 1-Cyclopropyl-3-propyl-8-(6-(((3-((2,2-dimethylpropanamido)methyl)phenyl)methyl)amino)pyridin-3-yl)xanthine:

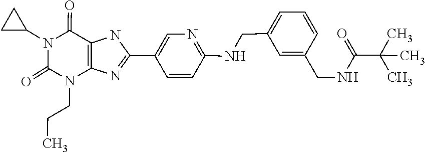

8-[6-({3-(Aminomethyl)benzyl}amino)pyridin-3-yl]-1-cyclopropyl-3-propylxanthine (1.0000 g, 2.2446 mmoles) and trimethylacetyl chloride (0.4060 g, 3.3669 mmoles) were combined in a flask with dry pyridine (15 mL). The reaction stirred at 25° C. for 24 hours. The reaction was checked for completion via HPLC. More trimethylacetyl chloride (10 uL) was added to the reaction, which stirred for an additional 24 hours. The solvent was removed in vacuo and methanol (3 mL) and water (30 mL) were added. The reaction was filtered and washed with water. The solid was collected and washed with DCM/MeOH, and refiltered to afford the product, 0.6500 g, 1.2273 mmoles, 54.68% yield. HPLC-MS conditions: 40%-80% MeOH (0.1% formic acid)/H₂O (0.1% formic acid) 10 min. 5 min. hold, Rt=10.645, LRMS ESI (M+H⁺) 530.45.

### EXAMPLE 32

### 1-Cyclopropyl-3-ethyl-8-(6-((4-(cyclopropylcarboxamidomethyl)benzyl)amino)pyridin-3-yl)xanthine:

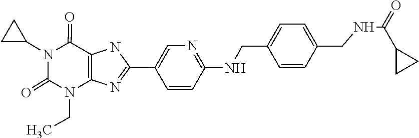

8-(6-((4-(Aminomethyl)benzyl)amino)pyridin-3-yl)-1-cyclopropyl-3-ethylxanthine (0.1500 g, 0.3477 mmoles) was combined with cyclopropanecarbonyl chloride (0.0947 mL, 1.0431 mmoles) in dry pyridine (20 mL). The reaction stirred at 25° C. for 24 hours. The solvent was removed in vacuo, and the reaction was filtered and washed with DCM to afford the product, 0.0582 g, 0.1165 mmoles, 33.50% yield. HPLC-MS conditions: 40%-80% MeOH (0.1% formic acid)/H₂O (0.1% formic acid) 10 min. 5 min. hold, Rt=10.753, LRMS ESI (M+H⁺) 500.40.

### EXAMPLE 33

### 1-Cyclopropyl-3-ethyl-8-(6-((4-(benzamidomethyl)benzyl)amino)pyrid-3-yl)xanthine:

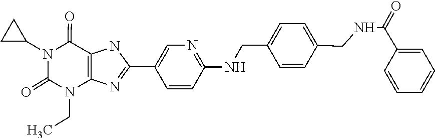

8-(6-((4-(Aminomethyl)benzyl)amino)pyridin-3-yl)-1-cyclopropyl-3-ethylxanthine (0.1500 g, 0.3477 mmoles) was combined with benzoyl chloride (0.1466 g, 0.1212 mL, 1.0431 mmoles) in dry pyridine (20 mL). The reaction stirred at 25° C. for 24 hours. The solvent was removed in vacuo, and the reaction was filtered and washed with DCM to afford the product, 0.1124 g, 0.2099 mmoles, 60.35% yield. HPLC-MS conditions: 40%-80% MeOH (0.1% formic acid)/H₂O (0.1% formic acid) 10 min. 5 min. hold, Rt=9.594, LRMS ESI (M+H⁺) 536.40.

### EXAMPLE 34

### 1-Cyclopropyl-3-propyl-8-(6-{[(4-{[(pyrrolidine-1-carbonyl)amino]methyl}phenyl)methyl]amino}pyridin-3-yl)xanthine:

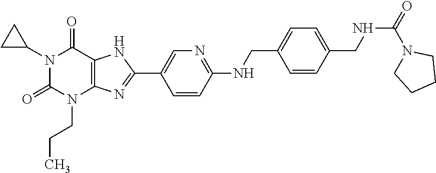 [SMILES: CCCn1c(=O)n(C2CC2)c(=O)c2[nH]c(-c3ccc(NCc4ccc(CNC(=O)N5CCCC5)cc4)nc3)nc21]

8-(6-((4-(Aminomethyl)benzyl)amino)pyridin-3-yl)-1-cyclopropyl propylxanthine (0.1000 g, 0.2245 mmoles) and pyrrolidine-1-carbonyl chloride (0.0450 g, 0.0372 mL, 0.3367 mmoles) were combined in a flask with dry pyridine (15 mL). The mixture stirred at 25° C. for 24 hours. The reaction was checked for completion via HPLC, and more pyrrolidine-1-carbonyl chloride (10 uL) was added. The reaction stirred for an additional 24 hours at 25° C. The solvent was removed in vacuo and methanol (˜20 mL) was added. The reaction was filtered and washed with methanol to afford the product, 0.0508 g, 0.0936 mmoles, 41.71% yield. HPLC-MS conditions: 40%-80% MeOH (0.1% formic acid)/H₂O (0.1% formic acid) 10 min. 5 min. hold, Rt=9.920, LRMS ESI (M+H⁺) 543.45.

### EXAMPLE 35

### 1-Cyclopropyl-3-ethyl-8-(6-{[(4-{[(pyrrolidine-1-carbonyl)amino]methyl}phenyl)methyl]amino}pyridin-3-yl)xanthine:

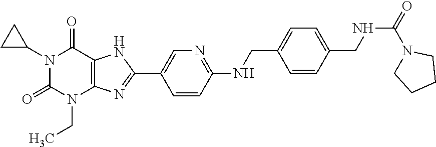 [SMILES: CCn1c(=O)n(C2CC2)c(=O)c2[nH]c(-c3ccc(NCc4ccc(CNC(=O)N5CCCC5)cc4)nc3)nc21]

8-[6-({[4-(Aminomethyl)phenyl]methyl}amino)pyridin-3-yl]-1-cyclopropyl-3-ethylxanthine (0.1000 g, 0.2318 mmoles) and pyrrolidine-1-carbonyl chloride (0.0464 g, 0.3476 mmoles) were combined in a flask with dry pyridine (15 mL). The reaction stirred at 25° C. for 24 hours. The reaction was checked for completion via HPLC, more pyrrolidine-1-carbonyl chloride (10 uL) was added, and stirring continued for an additional 24 hours. The solvent was removed in vacuo and methanol (˜20 mL) was added. The reaction was filtered and washed with methanol to afford the product, 0.0854 g, 0.1616 mmoles, 69.71% yield. HPLC-MS conditions: 40%-80% MeOH (0.1% formic acid)/H₂O (0.1% formic acid) 10 min. 5 min. hold, Rt=8.752, LRMS ESI (M+H⁺) 529.40.

### EXAMPLE 64

### 1-Cyclopropyl-3-propyl-8-(6-(((3-(((4-carboxyphenyl)formamido)methyl)phenyl)methyl)amino)pyridine-3-yl)xanthine:

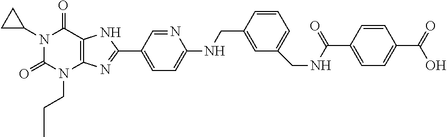 [SMILES: CCCn1c(=O)n(C2CC2)c(=O)c2[nH]c(-c3ccc(NCc4cccc(CNC(=O)c5ccc(C(=O)O)cc5)c4)nc3)nc21]

8-(6-((3-(Aminomethyl)benzyl)amino)pyridin-3-yl)-1-cyclopropyl-3-propylxanthine (0.5000 g, 1.1223 mmoles) and methyl 4-(chloroformyl) benzoate (0.2452 g 1.2345 mmoles) were combined in a flask with dry pyridine (˜15 mL). The reaction stirred at 25° C. for 24 hours. DCM (˜20 mL) was added to the reaction, which was then filtered and washed with DCM. The solid (0.1500 g, 0.2468 mmoles) was combined in an erlenmeyer flask with water (20 mL) and 1N NaOH (20 mL) and stirred at 25° C. for 2 hours. The pH was adjusted to 2 with 10% HCl. The solution was filtered and washed with water to afford the product, 0.1060 g, 0.1786 mmoles, 15.91% yield. HPLC-MS conditions: 40%-80% MeOH (0.1% formic acid)/H₂O (0.1% formic acid) 10 min. 5 min. hold, Rt=9.335, LRMS ESI (M+H⁺) 594.40.

Representative compounds of the present invention have been tested for their activity as A_{2A} and/or A_{2B} antagonists and shown to be active. Procedures similar to those described in Linden et al. (A_{2A}) (Molecular Pharmacology 2002, 61, 455-62) and Borrmann (A_{2B}) (J. Med. Chem. 2009, 52, 3994-4006) were used to test the activity of the compounds.

All references listed herein are individually incorporated in their entirety by reference.

Numerous modifications and variations of the present invention are possible in light of the above teachings. It is therefore to be understood that within the scope of the appended claims, the invention may be practiced otherwise than as specifically described herein.

What is claimed is:

1. A compound of Formula I or a stereoisomer or pharmaceutically acceptable salt thereof: 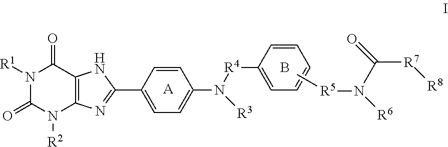
wherein:
Ring A is phenyl or a 5-6 membered heteroaryl having 1-3 heteroatoms selected from O, S, or N;
Ring B is phenyl or a 5-6 membered heteroaryl having 1-2 heteroatoms selected from O, S, or N;
R¹ is selected from: C₁₋₅ alkyl, —CH₂—C₂₋₄ alkenyl, —CH₂—C₂₋₄ alkynyl, C₃₋₆ cycloalkyl, and —C₂₋₅ alkylene-O—C₁₋₅ alkyl;
R² is selected from: C₁₋₅ alkyl, —CH₂—C₂₋₄ alkenyl, —CH₂—C₂₋₄ alkynyl, C₃₋₆ cycloalkyl, and —C₂₋₅ alkylene-O—C₁₋₅ alkyl;
R³ is selected from: H, C₁₋₅ alkyl, C₃₋₆ cycloalkyl, and —C₂₋₅ alkylene-O—C₁₋₅ alkyl;
R⁴ is —(CH₂)₁₋₆—;
R⁵ is —(CH₂)₁₋₆—;
R⁶ is selected from: H, C₁₋₅ alkyl, C₃₋₆ cycloalkyl, and —C₂₋₅ alkylene-O—C₁₋₅ alkyl;
R⁷ is absent or is selected from: C₁₋₅ alkyl, O, S, N—C₁₋₅ alkyl, and NH;
R⁸ is selected from: C₁₋₅ alkyl, —CH₂—C₂₋₄ alkenyl, —CH₂—C₂₋₄ alkynyl, C₃₋₆ cycloalkyl, —C₁₋₅ alkylene-O—C₁₋₅ alkyl, phenyl, 5-6 membered heterocycle, and 5-6 membered heteroaryl;
the phenyl and heteroaryl groups of R⁸ are optionally substituted with 1-3 groups independently selected from: C₁₋₄ alkyl, C₃₋₆ cycloalkyl, —C₁₋₃ alkylene-C₃₋₆ cycloalkyl, F, Cl, Br, I, —CN, OR^{a}, SR^{a}, NR^{a}R^{b}, CF₃, OCF₃, COR^{a}, CO₂R^{a}, C(O)NR^{a}R^{b}, OC(O)R^{a}, OCO₂R^{a}, OC(O)NR^{a}R^{b}, NR^{b}COR^{a}, NR^{b}CO₂R^{a}, NR^{b}C(O)NR^{a}R^{b}, and S(O)ₚNR^{a}R^{b};
each R^{a} is independently selected from: H, C₁₋₈ alkyl, C₃₋₆ cycloalkyl, and —C₁₋₃ alkylene-C₃₋₆ cycloalkyl;
each R^{b} is independently selected from: H, C₁₋₈ alkyl, C₃₋₆ cycloalkyl, and —C₁₋₃ alkylene-C₃₋₆ cycloalkyl;
alternatively, each NR^{a}R^{b} group is optionally selected from a 3-6 membered cyclic amine; and,
p is independently selected from: 0, 1, and 2.

2. A compound of claim 1, wherein the compound is of Formula IA: 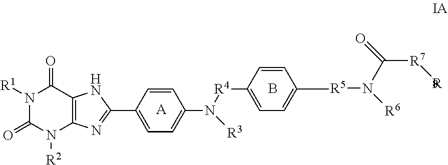
or a stereoisomer or pharmaceutically acceptable salt thereof.

3. A compound of claim 1, wherein the compound is of Formula IB: 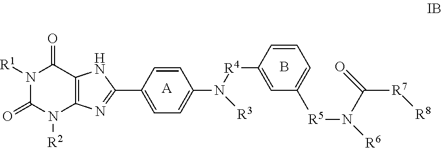
or a stereoisomer or pharmaceutically acceptable salt thereof.

4. A compound of claim 1, wherein the compound is of Formula IC: 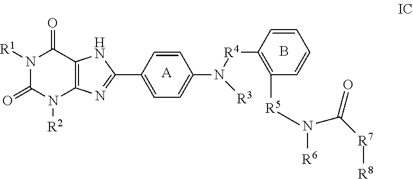
or a stereoisomer or pharmaceutically acceptable salt thereof.

5. A compound of claim 1, wherein the compound is of Formula II: 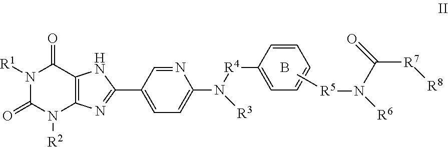
or a stereoisomer or pharmaceutically acceptable salt thereof.

6. A compound of claim 1, wherein the compound is of Formula IIA: 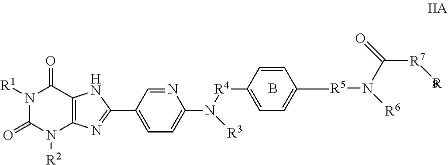
or a stereoisomer or pharmaceutically acceptable salt thereof.

7. A compound of claim 1, wherein the compound is of Formula IIB: 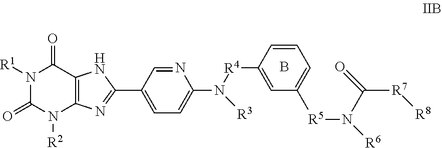
or a stereoisomer or pharmaceutically acceptable salt thereof.

8. A compound of claim 1, wherein the compound is of Formula IIC: 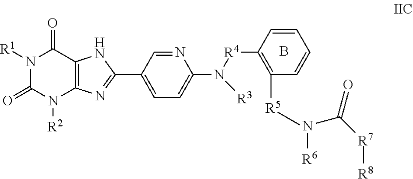
or a stereoisomer or pharmaceutically acceptable salt thereof.

9. A compound of claim 1, wherein the compound is of Formula III: 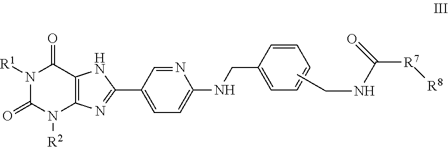
or a stereoisomer or pharmaceutically acceptable salt thereof.

10. A compound of claim 1, wherein the compound is of Formula IIIA: 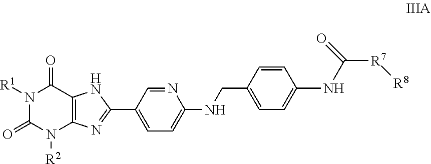
or a stereoisomer or pharmaceutically acceptable salt thereof.

11. A compound of claim 1, wherein the compound is of Formula IIIB: 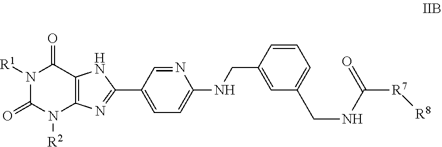
or a stereoisomer or pharmaceutically acceptable salt thereof.

12. A compound of claim 1, wherein the compound is of Formula IIIC: 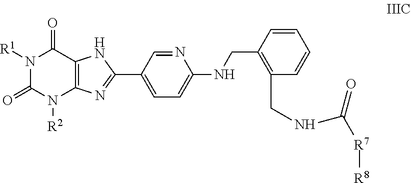
or a stereoisomer or pharmaceutically acceptable salt thereof.

13. A compound of claim 1, wherein:
Ring A is phenyl or a 6 membered heteroaryl having 1-2 heteroatoms selected from O, S, or N;
Ring B is phenyl or a 6 membered heteroaryl having 1 heteroatoms selected from N;
R¹ is selected from: C₁₋₃ alkyl, C₃₋₆ cycloalkyl, and —C₂₋₃ alkylene-O—C₁₋₂ alkyl;
R² is selected from: C₁₋₃ alkyl, C₃₋₆ cycloalkyl, and —C₂₋₃ alkylene-O—C₁₋₂ alkyl;
R³ is selected from: H and C₁₋₃ alkyl;
R⁴ is —(CH₂)₁₋₂—;
R⁵ is —(CH₂)₁₋₂—; and,
R⁶ is selected from: H and C₁₋₃ alkyl.

14. A compound of claim 1, wherein:
Ring A is phenyl or pyridyl;
Ring B is phenyl or pyridyl;
R¹ is selected from: ethyl, n-propyl, methoxyethylene, and cyclopropyl;
R² is selected from: ethyl, n-propyl, methoxyethylene, and cyclopropyl;
R³ is selected from: H and CH₃;
R⁴ is —(CH₂)₁₋₂—;
R⁵ is —(CH₂)₁₋₂—; and,
R⁶ is selected from: H and CH₃.

15. A compound of claim 1, wherein:
R⁷ is absent;
R⁸ is selected from: C₁₋₃ alkyl, C₃₋₆ cycloalkyl, —C₁₋₂ alkylene-O—C₁₋₂ alkyl, phenyl, and 5-6 membered heteroaryl;
the phenyl and heteroaryl groups of R⁸ are optionally substituted with 1-2 groups independently selected from: C₁₋₄ alkyl, —C₁₋₃ alkylene-C₃₋₆ cycloalkyl, F, Cl, OR^{a}, COR^{a}, and CO₂R^{a}; and,
each R^{a} is independently selected from: H and C₁₋₄ alkyl.

16. A compound of claim 1, wherein:
R⁷ is absent;
R⁸ is selected from: C₁₋₃ alkyl, C₃₋₄ cycloalkyl, —C₁ alkylene-O—C₁₋₂ alkyl, phenyl, thienyl, and pyridyl;
the phenyl and heteroaryl groups of R⁸ are optionally substituted with 1-2 groups independently selected from: C₁₋₄ alkyl, F, Cl, COR^{a}, and CO₂R^{a}; and,
each R^{a} is independently selected from: H and C₁₋₂ alkyl.

17. A compound of claim 1, wherein:
ring R⁸ is selected from phenyl, pyridyl, thienyl, furanyl, pyrrolyl, oxazolyl, isoxazolyl, thiazolyl, isothiazolyl, pyrimidyl, and pyridazinyl.

18. A compound of claim 1, wherein:
ring R⁸ is selected from phenyl, thienyl, and pyridyl.

19. A compound of claim 1, wherein the compound is selected from compounds 1-64 of Table 1 or pharmaceutically acceptable salt thereof.

20. A pharmaceutical composition, comprising: a therapeutically effective amount of a compound of claim 1 or a stereoisomer or pharmaceutically acceptable salt thereof and a pharmaceutically acceptable carrier.

21. A method for treating an adenosine A_{2A}-A_{2B} receptor associated state in a subject, comprising: administering to the subject an effective amount of a compound of claim 1 or a stereoisomer or pharmaceutically acceptable salt thereof.
